SEQUENCE LISTING (15 sequences)

SEQ ID NO: 1 - protein, 418 aa, Coronavirus SARS-Cov2 virus
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 48 aa, synthetic construct
  REGION 1..48: note = hybrid human chicken C4Bp
KKQGDADVCGEVAYIQSVVSDCHVPTAELRTLLEIRKLFLEIQKLKVE

SEQ ID NO: 3 - skipped

SEQ ID NO: 4 - protein, 7 aa, synthetic construct
  REGION 1..7: note = Postively charged peptide
GRRRRRS

SEQ ID NO: 5 - protein, 55 aa, synthetic construct
  REGION 1..55: note = OVX313
KKQGDADVCGEVAYIQSVVSDCHVPTAELRTLLEIRKLFLEIQKLKVEGRRRRRS

SEQ ID NO: 6 - protein, 475 aa, synthetic construct
  REGION 1..475: note = OVX033
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGSKKQGDADVCGEVAYIQSVVSDCHVPTAELRTLLEIRKLFLEIQKLKVEGRRRRRS

SEQ ID NO: 7 - DNA, 1257 bp, Coronavirus SARS-Cov2 virus
  source 1..1257: mol_type = genomic DNA
agcgacaatggcccgcaaaatcaacgcaacgcaccacgtatcactttcggtggccctagcgatagcacgggttctaatcagaatggtgaacgtagcggtgcacgcagcaagcaacgtcgcccgcagggcctcccgaacaatacggcgtcttggttcaccgcgctgacgcagcacggtaaggaagatctgaaatttccgcgtggtcagggtgttccgatcaatacgaatagctccccggacgatcagattggctattaccgtcgtgcgacccgtcgtattcgtggtggcgacggtaagatgaaagatctgtccccacgctggtatttctactacctgggtacgggccctgaggccggtctgccgtatggcgcgaacaaagacggcattatttgggtggcgaccgagggtgctctgaacacgccgaaagaccacatcggcacccgtaatccggcgaataacgcagcgatcgttttacaactgccgcaaggtacgactctgccgaagggtttttacgctgagggcagccgcggtggcagccaagcgagcagccgcagctcaagccgtagccgtaatagctccagaaactcgacgcctggcagctctcgtggcaccagcccggcacgtatggcaggcaacggtggcgatgctgcgctggccctgctgctgcttgatcgtctgaaccagctggagtccaagatgagcggtaagggtcagcagcagcaaggtcagaccgtgaccaagaaatctgccgcagaggcgagcaagaaaccgcgtcagaagcgtaccgcgaccaaggcctacaacgtcacccaagcgtttggtcgccgtggtccggagcagacgcaaggtaacttcggcgatcaagaactgatccgccaaggcaccgactacaagcactggccgcagatcgcgcaatttgcaccaagcgcgtcggcctttttcggtatgtcccgcattggtatggaagtgacgccgagcggtacctggctgacctataccggtgccatcaaactggatgataaagacccgaactttaaagatcaagtcattttgttgaacaagcatattgacgcatataagactttcccgccgaccgagccgaaaaaagacaaaaagaaaaaagccgatgaaacccaagcactgccgcagcgccaaaagaaacagcaaacggttaccttgctgccggctgcggacctggacgacttcagcaaacagttgcagcagagcatgagcagcgctgacagcacccaggcgtaa

SEQ ID NO: 8 - DNA, 144 bp, synthetic construct
  misc_feature 1..144: note = Hybrid human chicken C4Bp
  source 1..144: mol_type = other DNA
aagaaacagggtgatgctgacgtgtgcggcgaagtggcatatatccagagcgtcgtgagcgattgtcacgttccgacggcagagttgcgcacgctgttggaaatccgtaagctgttcttggagattcaaaagctcaaagttgag

SEQ ID NO: 9 - DNA, 21 bp, synthetic construct
  misc_feature 1..21: note = Postively charged peptide
  source 1..21: mol_type = other DNA
ggtcgtcgtcgcagacgttcc

SEQ ID NO: 10 - DNA, 165 bp, synthetic construct
  misc_feature 1..165: note = OVX313
  source 1..165: mol_type = other DNA
aagaaacagggtgatgctgacgtgtgcggcgaagtggcatatatccagagcgtcgtgagcgattgtcacgttccgacggcagagttgcgcacgctgttggaaatccgtaagctgttcttggagattcaaaagctcaaagttgagggtcgtcgtcgcagacgttcc

SEQ ID NO: 11 - DNA, 1428 bp, synthetic construct
  misc_feature 1..1428: note = Coding sequence of OVX033
  source 1..1428: mol_type = other DNA
agcgacaatggcccacagaatcaacgtaatgcaccgagaatcacctttggcggcccgagcgatagcacgggtagcaaccagaacggcgagcgcagcggtgcgcgttctaaacagcgccgtccgcaaggtctgccgaataacacggcgtcgtggtttacggcactgacccagcatggtaaggaagatttgaagtttccgcgtggccagggtgtcccgatcaataccaacagctcacctgatgaccagatcggctattatcgtcgcgccacgcgccgtatccgcggtggcgatggcaagatgaaagacttaagcccgcgttggtacttctattacctgggtacgggtccagaggctggtttgccgtatggtgctaacaaagacggcattatctgggtggcgaccgagggtgccctgaacaccccgaaagatcacattggtactcgtaaccctgcgaacaacgccgcgattgtgctgcaactgccgcagggtaccaccctgccgaagggtttctatgcagagggcagccgtggtggcagccaggcgagcagccgtagcagcagccgttcccgcaattctagccgcaatagcacgccgggttccagccgtggtacgtccccggcacgtatggcaggcaatggtggcgatgcggcactggcgctgctgttgctggaccgcctgaaccaactggagagcaagatgagcggcaagggtcagcagcagcaaggtcagactgtgaccaaaaagtccgcggcagaggccagcaagaaaccacgtcagaaacgtaccgcgaccaaagcgtacaatgttacccaggccttcggtcgccgtggccctgagcaaacccaaggtaatttcggtgaccaagaactgatccgccagggcacggactacaagcattggccgcagattgcgcagtttgcaccgagcgctagcgcgttcttcggtatgtcccgcattggtatggaagtgaccccgtctggcacctggctgacctacactggtgcaatcaaattggacgataaagacccgaactttaaagaccaagtcatcctcctgaacaagcacattgatgcgtacaagaccttcccgccgacggaaccgaaaaaagacaaaaagaaaaaagctgacgaaacccaagcattgccgcaacgtcaaaagaaacagcaaacggttaccctgcttccggcagcggatctggacgattttagcaagcaactgcaacaatctatgtcgagcgcggactccacccaggccggtagcaagaaacagggcgatgccgatgtctgcggcgaagttgcgtacattcagagcgtcgtgagcgactgtcacgttccgacggctgagctgcgtaccctgctggaaattcgtaagctgttcctggaaatccaaaagctgaaagttgagggtcgccgtcgtcgccgtagctaa

SEQ ID NO: 12 - DNA, 1425 bp, synthetic construct
  misc_feature 1..1425: note = Coding sequence of OVX030
  source 1..1425: mol_type = other DNA
agcgataatggcccacaaaatcaacgtaacgcaccaagaatcacctttggcggcccgagcgactccaccggttcgaatcagaatggtgagcgtagcggtgcacgcagcaagcaacgccgtccgcagggcttaccgaacaacacggcttcctggttcaccgcgctgactcagcacggtaaagaggacctgaagtttcctcgtggtcagggtgtgccgatcaacaccaacagctccccggatgaccagatcggttactatcgccgtgcaacccgccgtattcgtggtggcgacggtaagatgaaagatctgagcccgcgttggtatttctattacctgggtacgggtccggaagcgggcctgccgtacggtgcgaacaaagacggcatcatttgggttgcgaccgagggtgcgctgaatacgccgaaagatcacattggtacccgcaacccggctaataacgccgcgatcgtcttgcagctgccgcagggtaccaccctgccgaagggcttctatgccgagggtagccgcggtggcagccaagcgtctagccgtagcagcagccgtagccgtaatagctcccgtaatagcacgccgggtagcagccgtggcacgagcccggctcgcatggcaggcaatggcggcgatgcggcgcttgcactgctgctgctggaccgtctgaaccaactggagagcaagatgagcggcaagggccagcaacaacagggtcagaccgtcaccaagaaaagcgcagccgaggcgtctaaaaagccgcgtcagaagcgcacggcgacgaaagcatacaatgtgacgcaagcgttcggtcgtcgcggtccggagcagacccagggtaacttcggcgaccaagaactcatccgtcaaggtaccgattacaagcattggccacagattgcgcagtttgctccgagcgcttccgcatttttcggcatgtctcgtatcggtatggaagtgaccccgtccggtacctggctgacttatacgggtgccatcaaactggatgataaagatcctaactttaaagaccaagtgattttgttgaacaagcacattgacgcgtacaagaccttcccgccgaccgagccgaaaaaagacaagaaaaaaaaagccgatgaaacccaagcgctgccgcagcgccaaaagaaacaacaaactgtcacgctgctgcctgcggcggacctggacgatttttcgaagcaactgcaacagagcatgagcagcgcggactcaacccaggccggcagcaagaaacagggtgatgcagatgtttgtggcgaagtcgcctacattcagtctgttgtgagcgactgccatgttccgaccgcagagctgcgcacgctgttggaaattcgtaagctgttcttggaaatccagaaactgaaagttgagggtcgtcgtcgtcgccgcagc

SEQ ID NO: 13 - DNA, 1434 bp, synthetic construct
  misc_feature 1..1434: note = Coding sequence of OVX031
  source 1..1434: mol_type = other DNA
tctgataatggcccgcaatccaaccaacgttctgcaccgcgcatcactttcggtggcccgaccgacagcacggataacaaccagaacggtggccgcaacggtgcacgtccgaaacagcgtcgtccgcagggtctgccgaacaataccgcctcttggttcaccgcgctgacgcagcatggcaaggaagaactgcgtttcccgcgtggtcagggtgtaccgattaacacgaatagcggtcctgatgatcaaatcggctactatcgtcgtgcgacccgtcgtgtgcgcggtggcgacggcaagatgaaagaactgtccccacgctggtacttttactatctgggcaccggtccggaagcgtccctgccgtacggtgctaataaagagggcattgtttgggttgcgactgagggtgcgttgaacaccccgaaagaccacatcggtacccgtaatccgaacaataacgcagcgacggtgctgcaactgccgcagggcacgacgctgccgaagggtttctacgccgagggtagccgtggtggctcgcaggcgagcagccgctctagcagccgtagccgcggtaatagccgcaatagcacccctggcagcagccgtggtaattcccctgcgcgtatggcgagcggtggcggcgaaacggcgctggcgctgctcctgctggatcgtttgaaccagttggagagcaaggttagcggtaaaggccaacagcagcaaggtcaaaccgtcaccaaaaagtcagcagccgaggcaagcaaaaagccacgtcagaagcgcacggctacgaagcaatacaatgtcacccaagcctttggccgtcgcggtccggagcaaacgcaaggtaactttggcgatcaagacctgattcgccagggtaccgactataagcactggccgcaaatcgctcaattcgccccgagcgcctccgcgttcttcggtatgagccgcattggtatggaagtgaccccgagcggtacctggctgacctatcatggcgcgatcaaactggatgataaagacccgcagtttaaagacaacgttatcctgttaaacaagcacatcgatgcttacaagacctttccaccgaccgagccgaaaaaagacaaaaagaaaaagaccgacgaagcgcagccgctgccgcagcgccagaagaaacaaccgaccgttactcttctgccggccgcagacatggatgactttagccgccaactgcagaattcgatgagcggtgcaagcgcggatagcacccaggctggcagcaagaaacagggtgacgcagatgtgtgcggcgaagtcgcgtatattcaaagcgtggttagcgactgtcacgtgccgaccgcagagttgcgtacgctgctggaaattcgtaagctgttcctggaaattcagaaactgaaagtcgagggtcgtcgtcgtagacgtagc

SEQ ID NO: 14 - DNA, 1257 bp, synthetic construct; identical to SEQ ID NO: 7
  misc_feature 1..1257: note = Coding sequence of OVX032
  source 1..1257: mol_type = other DNA

SEQ ID NO: 15 - DNA, 1428 bp, synthetic construct
  misc_feature 1..1428: note = Coding sequence of OVX034
  source 1..1428: mol_type = other DNA
agcgacaatggcccgcaaaatcaacgcaacgcaccacgtatcactttcggtggccctagcgatagcacgggttctaatcagaatggtgaacgtagcggtgcacgcagcaagcaacgtcgcccgcagggcctcccgaacaatacggcgtcttggttcaccgcgctgacgcagcacggtaaggaagatctgaaatttccgcgtggtcagggtgttccgatcaatacgaatagctccccggacgatcagattggctattaccgtcgtgcgacccgtcgtattcgtggtggcgacggtaagatgaaagatctgtccccacgctggtatttctactacctgggtacgggccctgaggccggtctgccgtatggcgcgaacaaagacggcattatttgggtggcgaccgagggtgctctgaacacgccgaaagaccacatcggcacccgtaatccggcgaataacgcagcgatcgttttacaactgccgcaaggtacgactctgccgaagggtttttacgctgagggcagccgcggtggcagccaagcgagcagccgcagctcaagccgtagccgtaatagctccagaaactcgacgcctggcagctctcgtggcaccagcccggcacgtatggcaggcaacggtggcgatgctgcgctggccctgctgctgcttgatcgtctgaaccagctggagtccaagatgagcggtaagggtcagcagcagcaaggtcagaccgtgaccaagaaatctgccgcagaggcgagcaagaaaccgcgtcagaagcgtaccgcgaccaaggcctacaacgtcacccaagcgtttggtcgccgtggtccggagcagacgcaaggtaacttcggcgatcaagaactgatccgccaaggcaccgactacaagcactggccgcagatcgcgcaatttgcaccaagcgcgtcggcctttttcggtatgtcccgcattggtatggaagtgacgccgagcggtacctggctgacctataccggtgccatcaaactggatgataaagacccgaactttaaagatcaagtcattttgttgaacaagcatattgacgcatataagactttcccgccgaccgagccgaaaaaagacaaaaagaaaaaagccgatgaaacccaagcactgccgcagcgccaaaagaaacagcaaacggttaccttgctgccggctgcggacctggacgacttcagcaaacagttgcagcagagcatgagcagcgctgacagcacccaggcgggcagcaagaaacagggtgacgcagatgtgtgcggcgaagtcgcgtatattcaaagcgtggttagcgactgtcacgtgccgaccgcagagttgcgtacgctgctggaaattcgtaagctgttcctggaaattcagaaactgaaagtcgagggtcgtcgtcgtagacgtagctaa

(19) United States
(12) Patent Application Publication
(10) Pub. No.: US 2025/0121053 A1
(43) Pub. Date: Apr. 17, 2025

(54) VACCINE COMPOSITIONS AND THEIR USE

(71) Applicant: OSIVAX, Paris (FR)

(72) Inventors: Alexandre LE VERT, Paris (FR); Marjorie HALLER, Colombier Saugnieu (FR); Delphine GUYON-GELLIN, Saint Cyr Au Mont D'or (FR); Florence NICOLAS, Genas (FR)

(21) Appl. No.: 18/723,007

(22) PCT Filed: Dec. 16, 2022
(86) PCT No.: PCT/EP2022/086304

§ 371 Date: Jun. 21, 2024

(30) Foreign Application Priority Data
Dec. 22, 2021 (EP) 21306897.6
Jul. 22, 2022 (EP) 22306103.7

(51) Int. Cl. A61K 39/215; A61K 39/00; A61P 31/14; A61P 37/04; C07K 14/005

(52) U.S. Cl. CPC A61K 39/215; A61P 31/14; A61P 37/04; C07K 14/005; A61K 2039/70; C07K 2319/735; C12N 2770/20022; C12N 2770/20034

(57) ABSTRACT

The invention relates to immunogenic compositions and their use as a vaccine for the prevention of coronavirus disease in a human subject. More specifically, the invention relates to methods of use of an immunogenic composition in the prevention of coronavirus disease in a human subject in need thereof, said immunogenic composition comprising: a fusion protein comprising (i) a SARS-Cov2 nucleocapsid N antigen and, (ii) a carrier protein comprising a self-assembling polypeptide derived from C4bp oligomerization domain and a positively charged tail.

### CROSS REFERENCE TO RELATED APPLICATIONS

This application is a United States Application under 35 U.S.C. 371 claiming benefits of PCT Application No. PCT/EP2022/086304 filed on Dec. 16, 2022, which claims the benefit of priority from European Patent Application No. 22306103.7, filed on Jul. 22, 2022, and European Patent Application No. 21306897.6, filed on Dec. 22, 2021, the contents of each of which are incorporated herein by reference.

### REFERENCE TO AN ELECTRONIC SEQUENCE LISTING

The contents of the electronic sequence listing (OVX-03-US-Sequence Listing.xml; Size: 27,217 bytes; and Date of Creation: Jun. 21, 2024, is herein incorporated by reference in its entirety.

### TECHNICAL DOMAIN

The disclosure relates to immunogenic compositions and their use as a vaccine for the prevention of coronavirus disease in a human subject.

### BACKGROUND

At end of 2021, the global pandemic of coronavirus disease 2019 caused by the emerging severe acute respiratory syndrome coronavirus-2 (SARS-COV-2) had resulted in more than 260 million global cases with more than 5.2 million deaths. While some infected persons remain asymptomatic, some will develop a mild respiratory disease that will resolve with no or little medical attention, and others (10 to 20% of symptomatic persons), will experience a severe disease often occurring as a sudden deterioration ˜11 days after the onset of first symptoms (Chen, S., et al. 2020. Front. Med. 0. doi: 10.3389/fmed.2020.567296), associated with respiratory failure and multi-organ complications possibly leading to patients' deaths (“Interim Clinical Guidance for Management of Patients with Confirmed Coronavirus Disease (COVID-19)”. U.S. Centers for Disease Control and Prevention (CDC). 6 Apr. 2020. Archived from the original on 2 Mar. 2020, updated Feb. 12, 2021).

To date, vaccine development efforts have predominantly been aimed at the Spike(S) protein as the major antigen but there are concerns regarding “viral immune escape” since multiple mutations may enable the mutated virus strains to escape from immunity against S protein.

The nucleocapsid (N-protein) is relatively conserved among mutant strains of coronaviruses during spread and evolution.

Based on publicly available data at end of 2021, especially from WHO, on ongoing R&D efforts to develop COVID vaccines, there are currently around 25 programs that target the Nucleocapsid.

Within these programs the majority targets epitopes and only 8 programmes include the full-length Nucleocapsid antigen (2 DNA vaccines and 3 viral vector vaccines at clinical stage/1 bacterial vector vaccine and 2 viral vector vaccines at the preclinical stage) as shortly described hereafter:

### Clinical Stage Projects:

Genexine is developing GX-19N, a DNA vaccine encoding SARS-COV-2 receptor binding domain (RBD) foldon and nucleocapsid protein (NP) as well as plasmid DNA encoding SARS-CoV-2 spike protein currently in phase 2 (Ahn et al. 2021, https://doi.org/10.1101/2021.05.26.21257700).

Scancell is developing a bivalent DNA vaccine encoding both spike protein receptor-binding domain and full-length nucleocapsid protein of SARS-COV-2 (Brentville et al. 2021, https://doi.org/10.1101/2021.06.18.448932).

Vaxart is developing an oral tablet-based viral vector (Ad5) vaccine expressing full-length S and N antigens (presentation in a vaccine congress in May 2021: https://investors.vaxart.com/static-files/b555c11c-6863-4763-8539-12754840a652).

City of Hope is developing a synthetic Modified Vaccinia Ankara (sMVA) vector co-expressing the full-length SARS-COV-2 spike and nucleocapsid (Chiuppesi et al. 2020, https://doi.org/10.1038/s41467-020-19819-1).

ImmunityBio is developing a viral vector vaccine that expresses both the spike(S) protein modified to increase cell-surface expression (S-Fusion) and nucleocapsid (N) protein with an Enhanced T-cell Stimulation Domain (N-ETSD) to enhance MHC class I and II presentation and T-cell responses using a human adenovirus serotype 5 (hAd5) platform (Sieling et al, June 2021 https://doi.org/10.1101/2021.04.05.21254940, Rice et al, July 2021, https://pubmed.ncbi.nlm.nih.gov/34290317/).

### Preclinical Stage Projects:

Colorado State University is developing a replicating bacterium-vectored vaccine expressing SARS-COV-2 Membrane and Nucleocapsid proteins that demonstrated protection against severe COVID-19-like disease in hamsters (Jia, et al., 2021, https://doi.org/10.1038/s41541-021-00321-8).

University of Minnesota reported that a HAd5 expressing the nucleocapsid (N) protein leads to reduced weight loss and viral load, in both Syrian hamsters and k18-hACE2 mice (Matchett et al, https://doi.org/10.1101/2021.04.26.441518).

Northwestern University Feinberg School of Medicine reported that a Ad5 vector vaccine expressing SARS-COV-2 spike and nucleocapsid proteins would confer acute protection in both the lung and brain (Dangi et al, 2021, https://doi.org/10.1016/j.celrep.2021.109664).

Despite these developments, there is still a need to further develop novel coronavirus vaccines, in particular vaccines which would provide broad spectrum protection.

OVX836 (OSIVAX, Lyon, France) is a recombinant protein under clinical development as a vaccine against influenza strains. The antigenic part corresponds to the NP sequence of the A/WSN/1933 (H1N1) influenza virus. OVX836 protein contains 7 copies of NP, each fused to OVX313. The OVX313 sequence is derived from the C-terminal oligomerization domain of the human C4b binding protein (hC4BP) [Hofmeyer T, et al. J Mol Biol. 2013:425:1302-1317], but modified to minimize homology with the human sequence (hybrid chicken sequence; homology less than 20%).

The inventors now surprisingly found that similar fusion proteins with the nucleocapsid N protein of SARS-Cov2 strain can be efficiently produced, in particular in bacterial cell expression system, and provide significant protection against coronavirus disease such as SARS-Cov2 disease, including cross-protection to different coronavirus strains (broad spectrum), in particular as revealed in hamster studies.

### BRIEF DESCRIPTION

Thus, in one aspect, the present disclosure relates to an immunogenic composition for use as a vaccine for the prevention of coronavirus disease, in particular COVID-19, typically for protection from severe COVID-19, in a human subject in need thereof, wherein said immunogenic composition includes a fusion protein comprising:
(i) a nucleocapsid N antigen, preferably SARS-Cov2 N antigen, fused to
(ii) a carrier protein comprising a self-assembling polypeptide derived from C4bp oligomerization domain and a positively charged tail.

The disclosure also relates to a method for preventing, protecting, or cross-protecting from coronavirus disease, in particular COVID-19, typically for protecting from severe COVID-19, said method comprising administering, in a human subject in need thereof, an immunogenic amount of a fusion protein comprising
(i) a nucleocapsid N antigen, preferably SARS-Cov2 N antigen, fused to
(ii) a carrier protein comprising a self-assembling polypeptide derived from C4bp oligomerization domain and a positively charged tail.

The disclosure further relates to the use of a fusion protein comprising
(i) a nucleocapsid N antigen, preferably SARS-Cov2 N antigen, fused to
(ii) a carrier protein comprising a self-assembling polypeptide derived from C4bp oligomerization domain and a positively charged tail, in the manufacturing of a vaccine for preventing, protecting or cross-protecting from coronavirus disease, in particular COVID-19, typically for protecting from severe COVID-19.

In preferred embodiments of the above methods or use, said fusion is OVX033 as disclosed hereafter. In further preferred embodiments, said fusion protein is formulated as an immunogenic composition for intramuscular administration, and is administered intramuscularly.

Another aspect of the present disclosure relates to a fusion protein comprising
(i) a SARS-Cov2 nucleocapsid N antigen fused to
(ii) a carrier protein comprising a self-assembling polypeptide derived from C4bp oligomerization domain and a positively charged tail.

In specific embodiments, said fusion protein is not glycosylated, and/or is obtainable from bacterial expression system, for example E. coli expression system. In preferred embodiments, said fusion protein is OVX033, as encoded by SEQ ID NO:11.

Another aspect of the present disclosure relates to the nucleic acids encoding said fusion protein, in particular, a nucleic acid of SEQ ID NO:11, or an expression vector comprising a nucleic acid of SEQ ID NO:11, or a host cell, typically a bacterial host cell comprising a nucleic acid of SEQ ID NO:11.

### LEGENDS OF THE FIGURES

FIG. 1A: SDS PAGE Analysis of OVX030. Purified OXV030 was fractionated on 4-12% Bis-Tris electrophoresis gel under reducing and denaturing conditions and stained with Instant blue followed by a secondary anti-rabbit/HRP antibody (Sigma, A0545). SDS PAGE: 0.5, 1, 1.5 μg/well. MW: molecular weight marker.

FIG. 1B: Western Blot Analysis of OVX030. Purified OXV030 was fractionated on 4-12% Bis-Tris electrophoresis gel under reducing and denaturing conditions and blotted onto nitrocellulose membrane and probed with polyclonal rabbit anti-OVX313 followed by a secondary anti-rabbit/HRP antibody (Sigma, A0545). Western Blot: 150, 300, 600 ng/well. MW: molecular weight marker.

FIG. 1C: Western Blot Analysis of OVX030. Purified OXV030 was fractionated on 4-12% Bis-Tris electrophoresis gel under reducing and denaturing conditions and blotted onto nitrocellulose membrane and probed with polyclonal rabbit anti-OVX313 and anti-nucleocapsid antibody (Genetex, GTX135357) followed by a secondary anti-rabbit/HRP antibody (Sigma, A0545). Western Blot: 150, 300, 600 ng/well. MW: molecular weight marker.

FIG. 2A: Overexpression analysis of synthetic DNA constructs of N-OVX313 protein by SDS PAGE. Total fraction extract (normalized samples) was fractionated on 4-12% Bis-Tris electrophoresis gel under reducing and denaturating conditions and stained with Instant blue. BI: before induction. 032: Total fraction of Nucleocapsid protein from SARS-COV-2 overexpression. 030, 033, 034: synthetic DNA constructs of SARS-COV-2 Nucleocapsid protein fused to OligoDOM®. MW: molecular weight marker.

FIG. 2B: Overexpression analysis of synthetic DNA constructs of N-OVX313 protein by Western Blot. Total fraction extract (normalized samples) was fractionated on 4-12% Bis-Tris electrophoresis gel under reducing and denaturating conditions and blotted onto nitrocellulose membrane and probed with anti-nucleocapsid antibody (Genetex, GTX135357) followed by a secondary anti-rabbit/HRP antibody (Sigma, A0545). BI: before induction. 032: Total fraction of Nucleocapsid protein from SARS-COV-2 overexpression. 030, 033, 034: synthetic DNA constructs of SARS-COV-2 Nucleocapsid protein fused to OligoDOM®. MW: molecular weight marker.

FIG. 3A: Efficacy of OV033 in a hamster challenge model. Hamsters were vaccinated twice four weeks apart with 50 μg of OVX033 by the intramuscular (IM) or the intranasal route (IN) or NaCl as a negative control. Animals were challenged four weeks after the last vaccination with 10⁴ TCID₅₀ of B.1 (D614G) SARS-COV-2 strain. Body weight evolution post-infection (pi) is shown in FIG. 3A. Statistical analysis: * p<0.05, ** p<0.01

FIG. 3B: Hamsters were vaccinated twice four weeks apart with 50 μg of OVX033 by the intramuscular (IM) or the intranasal route (IN) or NaCl as a negative control. Animals were challenged four weeks after the last vaccination with 10⁴ TCID₅₀ of B.1 (D614G) SARS-COV-2 strain. Lung viral Titer 4 days post-infection is shown in FIG. 3B. Statistical analysis: * p<0.05.

FIG. 3C: Hamsters were vaccinated twice four weeks apart with 50 μg of OVX033 by the intramuscular (IM) or the intranasal route (IN) or NaCl as a negative control. Animals were challenged four weeks after the last vaccination with 10⁴ TCID₅₀ of B.1 (D614G) SARS-COV-2 strain. Area of pneumonia in the lungs 7 Days post infection is shown in FIG. 3C. Statistical analysis: ** p<0.01.

FIG. 4A: Efficacy of OV033 in a hamster challenge model with B.1 strain of SARS-Cov-2. Hamsters were vaccinated twice four weeks apart with 50 μg or 100 μg of OVX033 by the intramuscular (IM) or NaCl as a negative control. Animals were challenged four weeks after the last vaccination with 10⁴ TCID₅₀ of B.1 (D614G) SARS-COV-2 strain. Body weight evolution post-infection (pi) is shown in FIG. 4A. Statistical analysis: * p<0.05, ** p<0.01, *** p<0.001, **** p<0.0001.

FIG. 4B: Hamsters were vaccinated twice four weeks apart with 50 μg or 100 μg of OVX033 by the intramuscular (IM) or NaCl as a negative control. Animals were challenged four weeks after the last vaccination with 10⁴ TCID₅₀ of B.1 (D614G) SARS-COV-2 strain. Lung viral Titers 4 days post-infection are shown in FIG. 4B. Statistical analysis: * p<0.05.

FIG. 4C: Hamsters were vaccinated twice four weeks apart with 50 μg or 100 μg of OVX033 by the intramuscular (IM) or NaCl as a negative control. Animals were challenged four weeks after the last vaccination with 10⁴ TCID₅₀ of B.1 (D614G) SARS-COV-2 strain. Area of pneumonia in the lungs 7 Days post infection is shown in FIG. 4C. Statistical analysis: **** p<0.0001.

FIG. 5A: Efficacy of OV033 in a hamster challenge model with Delta strain of SARS-CoV-2. Hamsters were vaccinated twice four weeks apart with 50 μg of OVX033 or NaCl as a negative control by the intramuscular route (IM). Animals were challenged four weeks after the last vaccination with 10⁴ TCID₅₀ of B.1.617.2 SARS-COV-2 strain (Delta). Lung viral Titer 4 days post-infection is shown in FIG. 5A. Statistical analysis: ** p<0.01.

FIG. 5B: Hamsters were vaccinated twice four weeks apart with 50 μg of OVX033 or NaCl as a negative control by the intramuscular route (IM). Animals were challenged four weeks after the last vaccination with 10⁴ TCID₅₀ of B.1.617.2 SARS-COV-2 strain (Delta). Area of pneumonia in the lungs 7 Days post infection is shown in FIG. 5B. Statistical analysis: ** p<0.01.

FIG. 6A: Efficacy of OV033 in a hamster challenge model with Omicron strain of SARS-COV-2. Hamsters were vaccinated twice four weeks apart with 50 μg of OVX033 or NaCl as a negative control by the intramuscular route (IM). Animals were challenged four weeks after the last vaccination with 10⁴ TCID₅₀ of B. 1.1.529 SARS-COV-2 strain (Omicron). Lung viral Titer 4 days post-infection are shown in FIG. 6A. Statistical analysis: ** p<0.01.

FIG. 6B: Hamsters were vaccinated twice four weeks apart with 50 μg of OVX033 or NaCl as a negative control by the intramuscular route (IM). Animals were challenged four weeks after the last vaccination with 10⁴ TCID₅₀ of B.1.1.529 SARS-COV-2 strain (Omicron). Viral titer 7 days post-infection in nasal turbinates are shown in FIG. 6B. Statistical analysis: ** p<0.01.

FIG. 6C: Hamsters were vaccinated twice four weeks apart with 50 μg of OVX033 or NaCl as a negative control by the intramuscular route (IM). Animals were challenged four weeks after the last vaccination with 10⁴ TCID₅₀ of B.1.1.529 SARS-COV-2 strain (Omicron). Area of pneumonia in the lungs 7 Days post infection are shown in FIG. 6C. Statistical analysis: ** p<0.01.

FIG. 7A: Immunogenicity of OV033 in hamster. Hamsters were vaccinated twice four weeks apart with 50 μg of OVX033 or NaCl as a negative control by the intramuscular route (IM). Animals were euthanized four weeks after the last vaccination and immune responses were characterized by ELISA. N-specific serum IgG antibody titers in sera at D56_is shown in FIG. 7A. Statistical analysis: * p<0.01.

FIG. 7B: Hamsters were vaccinated twice four weeks apart with 50 μg of OVX033 or NaCl as a negative control by the intramuscular route (IM). Animals were euthanized four weeks after the last vaccination and immune responses were characterized by IFNγ ELISpot. IFNγ ELISpot responses in spleen at D56 are shown in FIG. 7B. Statistical analysis: ** p<0.01.

FIG. 7C: Hamsters were vaccinated twice four weeks apart with 50 μg of OVX033 or NaCl as a negative control by the intramuscular route (IM). Animals were euthanized four weeks after the last vaccination and immune responses were characterized by IFNγ ELISpot. IFNγ ELISpot responses in lungs at D56 are shown in FIG. 7C. Statistical analysis: ** p<0.01.

FIG. 8: Mean Number of NP-specific IFNγ spot forming T-cells (SFC/2×10⁵ PBMC) for each of the group (OVX032, OVX030, OVX30+IFA, OVX030+AdaVax), 8 days after the second vaccination. Statistics were performed using the Kruskal-Wallis statistic test followed by the Dunn's multiple comparisons test *** p<0.001.

### DETAILED DESCRIPTION

### Definitions

In order that the present disclosure may be more readily understood, certain terms are first defined. Additional definitions are set forth throughout the detailed description.

The term “amino acid” refers to naturally occurring and unnatural amino acids (also referred to herein as “non-naturally occurring amino acids”), e.g., amino acid analogues and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogues refer to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogues can have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function similarly to a naturally occurring amino acid. The terms “amino acid” and “amino acid residue” are used interchangeably throughout.

Substitution refers to the replacement of a naturally occurring amino acid either with another naturally occurring amino acid or with an unnatural amino acid.

As used herein, the term “protein” refers to any organic compounds made of amino acids arranged in one or more linear chains (also referred as “polypeptide”) and folded into a globular form. It includes proteinaceous materials or fusion proteins. The amino acids in such polypeptide chain may be joined together by the peptide bonds between the carboxyl and amino groups of adjacent amino acid residues. The term “protein” further includes, without limitation, peptides, single chain polypeptide or any complex proteins consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation, PASylation and the like, incorporation of non-natural amino acids, amino acid modification for chemical conjugation or other molecule, etc. . . . .

The term “recombinant protein”, as used herein, includes proteins that are prepared, expressed, created or isolated by recombinant means, such as fusion proteins isolated from a host cell transformed to express the corresponding protein, e.g., from a transfectoma, etc. . . . .

As used herein, the term “fusion protein” refers to a recombinant protein comprising at least one polypeptide chain which is obtained or obtainable by genetic fusion, for example by genetic fusion of at least two gene fragments encoding separate functional domains of distinct proteins. A protein fusion of the present disclosure includes for example at least a coronavirus nucleocapsid N antigen and at least one other moiety, the other moiety being a carrier protein comprising a self-assembling polypeptide derived from C4bp oligomerization domain and a positively charged tail thereof as described below.

As used herein, the term “antigenic” polypeptide includes immunogenic fragments and epitopes of a particular polypeptide (for example the nucleocapsid N of coronavirus strain) capable of inducing an immune response against such antigenic polypeptides (for example N-specific immune response), at least when such antigenic polypeptide is fused to the carrier protein as disclosed herein.

As used herein, the percent identity between the two sequences is a function of the number of identical positions shared by the sequences (i. e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the Needleman and Wunsch algorithm (NEEDLEMAN, and Wunsch).

The percent identity between two nucleotide or amino acid sequences may also be determined using for example algorithms such as EMBOSS Needle (pair wise alignment; available at www.ebi.ac.uk, Rice et al 2000 Trends Genet 16:276-277). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification.

As used herein, the term, “optimized” means that a nucleotide sequence has been altered to encode an amino acid sequence using codons that are preferred in the production cell or organism, generally a bacterial cell, for example, Escherichia coli bacterial strain. The optimized nucleotide sequence is engineered to retain completely or as much as possible the amino acid sequence originally encoded by the starting nucleotide sequence. The amino acid sequences encoded by optimized nucleotide sequences are also referred to as optimized.

As used herein, the term “subject” includes any human or nonhuman animal. The term “nonhuman animal” preferably includes mammals, such as nonhuman primates, sheep, dogs, cats, horses, etc.

As used herein, a “variant” of a polypeptide may be natural or artificial mutant variants, for example obtained typically by amino acid substitution, deletion or insertion as compared to the corresponding native polypeptide. In certain embodiments, a variant may have a combination of amino acid deletions, insertions or substitutions throughout its sequence, as compared to the parent polypeptide.

In the context of the present disclosure, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn A, C, D, E, G, H, K, N, Q, R, S, P, and formation T
Flexible residues Q, T, K, S, G, P, D, E, and R

A “functional variant” is a variant which retains the properties of interest of the native polypeptide.

In preferred embodiments, a variant comprises an amino acid sequence which is at least 50%, 60%, 70%, 80%, 90%, or 95% identical to the native polypeptide sequence.

As such, polypeptides containing substitutions, insertions and/or additions, deletions and covalent modifications with respect to reference sequences, in particular the nucleocapsid N fusion proteins disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

As used herein, the term “coronavirus disease” refers to any disease caused by coronavirus infection, and in particular, SARS (severe acute respiratory syndrome), MERS (middle east respiratory syndrome) or, preferably, COVID-19 (coronavirus disease 2019), and more specifically severe COVID-19.

As used herein, the term “prevent” or “preventing” in relation to a disease refers to one or more of (1) reducing the risk of experiencing or displaying the disease condition or disorder or any symptoms associated to the disease, in an individual who may be exposed at such risk, and (2) reducing the risk of severe disease, or of exhibiting one or more severe symptoms of the disease or (3) reducing the viral shedding, thereby potentially reducing the spread of the virus from one subject to another. In particular, with reference to the prevention of an infectious disorder, the term “prevention” may refer to the prevention of infection by the infectious agent, inhibition of the replication of the infectious agent, reduction or eliminating any symptoms associated to the disease, reduction of viral shedding, or reduction of the severity and/or the duration of one or more of the symptoms associated to the infection, or eradication of the infectious agent. As used herein, when referring to reducing the incidence of severe disease or of exhibiting one or more severe symptoms of the disease, the term “protection” may be used interchangeably.

For example, in relation to a coronavirus disease, a severe coronavirus disease may refer to (i) Individuals who have SpO2<94% on room air at sea level, a ratio of arterial partial pressure of oxygen to fraction of inspired oxygen (PaO2/FiO2)<300 mm Hg, a respiratory rate >30 breaths/min, or lung infiltrates >50%; and/or, (ii) Individuals who have respiratory failure, septic shock, and/or multiple organ dysfunction. In some embodiments, the term “severe disease” with respect to a coronavirus disease refers to individuals with respiratory deficiency symptoms requiring hospitalization.

Prevention or protection is determined in a group of subjects and may not be applicable to all individuals. Typically, prevention or protection may be determined by randomized clinical trials as compared to a control group.

### The Nucleocapsid N Antigen

As used herein, the term “nucleocapsid N antigen” or simply “N antigen” refers to any natural coronavirus nucleocapsid N protein or their antigenic variants.

Natural coronavirus nucleocapsid N proteins include, without limitation, the nucleocapsids of any of the coronavirus, in particular, SARS-Cov1, MERS-Cov and SARS-Cov2.

In some embodiments, the nucleocapsid antigen (N) is derived from viral strain of SARS-Cov2, more specifically from a SARS-Cov2 strain selected from the group consisting of Wuhan (A), Europe (B.1), Alpha-UK (B.1.1.7), Beta S. Africa (B.1.351), Gamma Brazil (P.1), Delta India (B.1.617.2) and Omicron strain.

In specific embodiments, the nucleocapsid antigen (N) is the N antigen of SARS-Cov2 strain, more specifically, from strain Wuhan (A), comprising the polypeptide of SEQ ID NO:1.

In specific embodiments, an antigenic variant is a fragment of coronavirus nucleocapsid antigen having at least 50, 100, 150, 200, 250, 300, 350, 400, 450, 490 consecutive amino acid residues of the wild type coronavirus nucleocapsid of coronavirus SARS-Cov1, MERS-Cov or SARS-Cov2, preferably derived from SEQ ID NO:1. A fragment of coronavirus nucleocapsid antigen is by definition at least one amino acid shorter than corresponding full length natural coronavirus nucleocapsid, typically from SARS-Cov1, MERS-Cov or SARS-Cov2.

In specific embodiments, an antigenic variant of coronavirus nucleocapsid antigen is an antigenic polypeptide variant having at least 50%, 60%, 70%, 80%, 90%, 95% or 99% identity to corresponding wild-type sequence of a nucleocapsid of coronavirus, typically from SARS-Cov1, MERS-Cov or, preferably, SARS-Cov2. Preferably, an antigenic variant of coronavirus nucleocapsid antigen is an antigenic polypeptide variant having at least 50%, 60%, 70%, 80%, 90%, 95% or 99% identity to SEQ ID NO:1.

In a particular embodiment, said variant differs from the corresponding coronavirus nucleocapsid native antigen, through only amino acid substitutions, with natural or non-natural amino acids, preferably only 1, 2, 3, 4, 5, 6, 7, 8, 9 or 10 amino acid substitutions with natural amino acids, in particular as compared to the native SARS-Cov2 nucleocapsid antigen of SEQ ID NO: 1. In a specific embodiment, a variant is a mutant variant having 1, 2 or 3 amino acid substitutions with natural amino acids as compared to the native SARS-Cov2 nucleocapsid antigen of SEQ ID NO:1. In more specific embodiment, said variant is a natural variant (i.e. a variant found in nature which does not result from human recombinant technology) as compared to the SARS-Cov2 nucleocapsid antigen of SEQ ID NO:1 with only 1, 2, 3, 4, 5, 6, 7, 8, 9 or 10 amino acid substitutions as compared to SARS-Cov2 nucleocapsid antigen of SEQ ID NO:1.

In more specific embodiments, the amino acid sequence of said mutant variant may differ from the native coronavirus nucleocapsid antigen through mostly conservative amino acid substitutions; for instance at least 10, such as at least 9, 8, 7, 6, 5, 4, 3, 2 or 1 of the substitutions in the variant are conservative amino acid residue replacements.

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also may be substantially retained in a variant mutant polypeptide as compared to a parent polypeptide of any coronavirus nucleocapsid antigen, typically of SEQ ID NO:1.

In specific embodiments, a mutant variant comprises a polypeptide which is identical to SEQ ID NO: 1, except for 1, 2 or 3 amino acid residues which have been replaced by another natural amino acid by conservative amino acid substitutions as defined above.

In specific embodiments, a variant of the coronavirus nucleocapsid antigen does not comprise any mutation as compared to the parent polypeptide of SEQ ID NO:1 in the epitope recognized by the human immune system as described for example in IEDB database (immune epitope data base) accessible under www.IEDB.org. In specific embodiments, a variant of the coronavirus nucleocapsid does not include a mutation, as compared to the parent polypeptide of SEQ ID NO:1, in any of conserved amino acid residues between the nucleocapsid of SEQ ID NO: 1 (from strain Wuhan (A)), and one of Europe (B.1), Alpha-UK (B.1.1.7), Beta S. Africa (B.1.351), Gamma Brazil (P.1) Delta India (B.1.617.2) and Omicron, corresponding nucleocapsid amino acid sequence.

As used herein, “conserved amino acid residues” correspond to the amino acid residues which are identical between nucleocapsids of strains Wuhan (A) and one of Europe (B.1), Alpha-UK (B.1.1.7), Beta S. Africa (B.1.351), Gamma Brazil (P.1) Delta India (B.1.617.2) and Omicron nucleocapsid amino acid sequences, when aligned using standard sequence protein alignments such as those using BLAST algorithm.

### The Carrier Protein

As used herein, the term “carrier protein” designates generally a protein to which antigens are conjugated or fused and thereby rendered more immunogenic. The function of the carrier protein is to increase the immunogenicity of said antigen to which it is conjugated or fused.

The carrier protein for use in the fusion protein comprises a self-assembling polypeptide derived from C4bp oligomerization domain and a positively charged tail. The complement inhibitor C4-binding protein (C4bp) is an abundant plasma protein first discovered in mice. Its natural function is to inhibit the classical and lectin pathways of complement activation. The last exon of the C4bp alpha chain gene encodes the only domain in the protein which does not belong to the complement control protein family. This non-complement control protein domain contains 57 amino acid residues in human and 54 amino acid residues in mice and is both necessary and sufficient for the oligomerization of the C4bp. It has been found that, when fused to antigens, said self-assembling polypeptide is also necessary and sufficient for the oligomerization of the resulting fusion protein.

PCT/IB2004/002717 and PCT/EP03/08926 describe the use of mammalian C4bp oligomerization domains to increase the immunogenicity of antigens in mammals. WO2007/062819 further describe a C4bp oligomerization domain of chicken species and variants thereof.

In preferred embodiments, in order to minimize self-immune reaction, the self-assembling polypeptide has an identity to human C4bp oligomerization domain which is lower than 30%, preferably lower than 20%.

In particular, in specific embodiments, said self-assembling polypeptide derived from C4bp oligomerization domain comprises or essentially consists of SEQ ID NO:2.

In specific embodiments, a functional variant of the self-assembling polypeptide has at least 50%, 60%, 70%, 80%, 90%, 95% or 99% identity to SEQ ID NO:2.

A functional variant may include any variant with one or more amino acid additions, deletions and/or substitutions as compared to SEQ ID NO:2 which retain the self-assembling property of the polypeptide of SEQ ID NO:2.

In a particular embodiment, said variant differs from SEQ ID NO:2, through only amino acid substitutions, with natural or non-natural amino acids, preferably only 1, 2, 3, 4, 5, 6, 7, 8, 9 or 10 amino acid substitutions with natural amino acids. In a specific embodiment, a variant is a mutant variant having 1, 2 or 3 amino acid substitutions with natural amino acids as compared to SEQ ID NO:2.

In more specific embodiments, the amino acid sequence of said mutant variant may differ from the self-assembling polypeptide of SEQ ID NO:2 through mostly conservative amino acid substitutions; for instance, at least 10, such as at least 9, 8, 7, 6, 5, 4, 3, 2 or 1 of the substitutions in the variant are conservative amino acid residue replacements.

The carrier protein further comprises a C-terminal tail consisting of positively charged peptide. The C-terminal tail is preferably a peptide consisting of 6-10 amino acids, with at least 50% of positively charged amino acids. Amino acids with positive charges include arginine or lysine. Examples of such positively charged peptide are disclosed in WO2014/090905 and WO2014/147087.

In preferred embodiments, said positively charged tail comprises the sequence ZXBBBBZ (SEQ ID NO:3), wherein (i) Z is absent or is any amino acid, (ii) X is any amino acid, and (iii) B is an arginine or a lysine, preferably said positively charged tail comprises or essentially consists of the sequence of SEQ ID NO:4.

In more preferred embodiments, said carrier protein essentially consists of OVX313 polypeptide, corresponding to the polypeptide of SEQ ID NO:5.

In specific embodiments, said carrier protein is a functional variant of OVX313 polypeptide of SEQ ID NO:5 having at least 70%, 80%, or more preferably at least 90% identity to SEQ ID NO: 5.

In other embodiments, said carrier protein is a functional variant of OVX313 polypeptide of SEQ ID NO:5 which differ from SEQ ID NO:5, by only 1, 2, 3, 4, 5, 6, 7, 8, 9, or 10 amino acids by amino acid substitution. In other embodiments, said carrier protein is a functional variant of OVX313 polypeptide of SEQ ID NO:5 which differ from SEQ ID NO:5, by only 1, 2, 3, 4, 5, 6, 7, 8, 9, or 10 amino acids by conservative amino acid substitution.

### The N Fusion Protein

The fusion protein for use according to the present disclosure comprises
(i) a coronavirus nucleocapsid antigen, as defined above, and,
(i) a carrier protein, as defined above, comprising a self-assembling polypeptide derived from C4bp oligomerization domain and a positively charged tail,

The resulting fusion protein with nucleoprotein antigen is called hereafter for ease of reading the “N fusion protein”.

In specific embodiments, the carrier protein is fused C-terminally to the nucleocapsid antigen, optionally via a peptide linker. Peptide linker may be any short peptide linker generally used for fusion protein. Preferred peptide linkers, includes glycine-serine linker, such as the dipeptide gly-ser, or gly-ser-ser-ser, or (gly-ser-ser-ser)n, wherein n is an integer between 1 and 4.

In specific embodiments, said N fusion protein forms heptameric particles after self-assembling.

In specific embodiments, said N fusion protein form particles with diameters below 100 nm, for example between 5 and 100 nm, and more specifically between 10 and 100 nm, after self-assembling. The diameter of said particle may be measured for example by dynamic light scattering (DLS). DLS measures the hydrodynamic diameter of particles across the size range of approximatively 0.3 nm to 10 μm. DLS measurements are very sensitive to temperature and dispersant viscosity. Therefore, the temperature must be kept constant at 25° C. and the viscosity of the dispersant must be known.

In specific embodiments, said N fusion protein comprises the nucleocapsid N selected from SARS-Cov2 strains, preferably selected from the group consisting of Wuhan (A), Europe (B.1), Alpha-UK (B.1.1.7), Beta S. Africa (B.1.351), Gamma Brazil (P.1), Delta India (B.1.617.2) and Omicron. In preferred embodiments, said N fusion protein comprises the nucleocapsid N selected from Wuhan (A) strain.

In more preferred embodiments, said N fusion protein essentially consists of OVX033 polypeptide, corresponding to the polypeptide of SEQ ID NO:6, and more preferably encoded by SEQ ID NO:11.

In specific embodiments, said N fusion protein is a functional variant of OVX033 polypeptide having at least 70%, 80%, or more preferably at least 90% identity to SEQ ID NO:6.

In other embodiments, said N fusion protein is a functional variant of OVX033 polypeptide of SEQ ID NO:6 which differ from SEQ ID NO:6, by only 1, 2, 3, 4, 5, 6, 7, 8, 9, or 10 amino acids by amino acid substitution. In other embodiments, said N fusion protein is a functional variant of OVX033 polypeptide which differ from SEQ ID NO:6, by only 1, 2, 3, 4, 5, 6, 7, 8, 9, or 10 amino acids by conservative amino acid substitution.

The inventors have surprisingly found that such N fusion proteins have excellent immunogenic properties in particular for protecting from coronavirus disease, in particular when produced in a bacterial expression system, such as, E. coli expression systems.

In specific embodiments, the N fusion protein of the present disclosure does not exhibit N-glycosylation typical of glycoproteins produced in mammalian cells (such as CHO or human cell lines). In more specific embodiments, the N fusion protein is not glycosylated. In other specific embodiments, the N fusion proteins of the present disclosure is obtainable or obtained from a bacterial expression system, for example E. coli expression system.

### Methods for Preparing the N Fusion Protein

The N fusion protein for use according to the present disclosure may be prepared by any conventional methods for preparing recombinant proteins, using nucleic acid molecules that encode said N fusion protein which nucleotide sequence can be easily derived using the genetic code and, optionally taking into account the codon bias depending on the host cell species.

Preferred examples of nucleotide sequence which can be used to prepare the N fusion proteins are those encoding the amino acid sequences of SEQ ID NO: 1-6, typically as described in Tables 2 and 3.

The nucleic acid molecules may derive from the latter sequences and be optimized for protein expression in prokaryotic cells, for example, in E. coli bacterial cells.

In more preferred embodiments, said N fusion protein is prepared in a bacterial cell expression system using an optimized coding nucleic acid sequence of SEQ ID NO:11. The inventors surprisingly found that the resulting N fusion protein is produced with suitable quality and yield with such optimized sequence as compared with other nucleotide sequences.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the disclosure can be, for example, DNA or RNA and may or may not contain intronic sequences. In an embodiment, the nucleic acid may be present in a vector such as a recombinant plasmid vector.

Nucleic acids can be obtained using standard molecular biology techniques. Once DNA fragments encoding the nucleoprotein antigen, are obtained, these DNA fragments can be further manipulated by standard recombinant DNA techniques. In these manipulations, a DNA fragment for example encoding the nucleocapsid antigen may be operatively linked to another DNA molecule, for example a fragment encoding the carrier protein and optionally a linker.

The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The N fusion proteins of the present disclosure (in particular OVX033) can then be produced in a host cell transfectoma using, for example, a combination of recombinant DNA techniques and gene transfection methods as is well known in the art.

For example, to express the N fusion protein (typically OVX033), corresponding fragments thereof, DNAs encoding partial or full-length recombinant proteins can be obtained by standard molecular biology or biochemistry techniques (e.g., DNA chemical synthesis, PCR amplification or cDNA cloning) and the DNAs can be inserted into expression vectors such that the genes are operatively linked to transcriptional and translational control sequences.

In this context, the term “operatively linked” is intended to mean that a coding polypeptide sequence is ligated into a vector such that transcriptional and translational control sequences within the vector serve their intended function of regulating the transcription and translation of the recombinant N fusion protein. The expression vector and expression control sequences are chosen to be compatible with the expression host cell used. The protein encoding genes are inserted into the expression vector by standard techniques.

The recombinant expression vector can encode a signal peptide that facilitates secretion of the recombinant fusion protein from a host cell. The N fusion protein encoding gene can be cloned into the vector such that the signal peptide is linked in frame to the amino terminus of the recombinant protein. The signal peptide can be the native signal peptide of C4bp or a heterologous signal peptide (i.e., a signal peptide from a non-C4bp protein). In specific embodiments, the signal peptide is the methionine amino acid.

In addition to the N fusion protein encoding sequences, the recombinant expression vectors disclosed herein may carry regulatory sequences that control the expression of the recombinant fusion protein in a host cell. The term “regulatory sequence” is intended to include promoters, enhancers and other expression control elements (e.g., polyadenylation signals) that control the transcription or translation of the protein encoding genes. It will be appreciated by those skilled in the art that the design of the expression vector, including the selection of regulatory sequences, may depend on such factors as the choice of the host cell to be transformed, the level of expression of protein desired, etc.

In specific embodiments, the expression vector comprises the coding sequence of SEQ ID NO: 11 operatively linked to a bacterial promoter for expression in a bacterial host cell.

Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1.

In addition to the N fusion protein encoding sequences and regulatory sequences, the recombinant expression vectors of the present disclosure may carry additional sequences, such as sequences that regulate replication of the vector in host cells (e.g., origins of replication) and selectable marker genes. The selectable marker gene facilitates selection of host cells into which the vector has been introduced. For example, the selectable marker gene confers resistance to antibiotics (in bacterial expression system) or drugs (such as G418).

For expression of the N fusion proteins, the expression vector(s) encoding the recombinant protein is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the proteins of the present disclosure in either prokaryotic or eukaryotic host cells.

Expression of the N proteins may be carried out in prokaryotic cells, for example E. coli host cell. The N fusion protein may then be recovered by lysis of the bacterial cells, and further purification using standard purification procedures. In specific embodiments, the N fusion protein is produced according to the method similar to the one disclosed in DelCampo 2021 (Frontiers in Immunology, doi: 10/3389/fimm.2021.678483).

In specific embodiments, the disclosure relates to a bacterial host cell, typically E. coli host cell, for the production of the N fusion protein, comprising the coding sequence of SEQ ID NO: 11 or an expression vector comprising the coding sequence of SEQ ID NO:11.

### Immunogenic Compositions

In another aspect, the present disclosure provides a composition, e.g. an immunogenic composition containing an N fusion protein as described in the previous sections, and one or more pharmaceutically acceptable excipients.

The immunogenic composition includes any aqueous vehicle suitable for a parenteral, intranasal, intramuscular, or subcutaneous administration (e.g., by intramuscular injection). These may be in particular isotonic, sterile, saline solutions (monosodium or disodium phosphate, sodium, potassium, calcium or magnesium chloride and the like or mixtures of such salts).

In specific embodiments, said N fusion protein comprises at least 400 amino acid residues, for example between 400 and 600 amino acid residues, for example between 540 and 560 amino acid residues, optionally, said N fusion protein forms particles with diameters between 5 and 100 nm, for example between 10 and 100 nm in said immunogenic compositions, as disclosed herein.

For example, said immunogenic composition is an aqueous composition which comprises a polypeptide of SEQ ID NO:6 (OVX033) or a variant having at least 70%, 80%, preferably at least 90%, or at least 95% identity to SEQ ID NO:6, formulated together with one or more pharmaceutically acceptable excipients.

In specific embodiments, said immunogenic composition is an aqueous composition which comprises a polypeptide of SEQ ID NO:6 (OVX033), which polypeptide has been obtained from SEQ ID NO:11 coding sequence, and is not glycosylated.

In preferred embodiments, said immunogenic composition is an aqueous composition which comprises a polypeptide of SEQ ID NO:6 (OVX033), which polypeptide has been obtained from SEQ ID NO:11 coding sequence, and is obtained from a bacterial expression system, typically E. coli host cell.

In specific embodiments, said immunogenic composition provides a fusion protein comprising the nucleocapsid N selected from SARS-Cov2 strains selected from the group consisting of Wuhan (A), Europe (B.1), Alpha-UK (B.1.1.7), Beta S. Africa (B.1.351), Gamma Brazil (P.1), Delta India (B.1.617.2) and Omicron.

In specific embodiments, said immunogenic composition provides a plurality of fusion proteins from different SARS-Cov2 strains, in particular, but not necessarily, selected from the group consisting of Wuhan (A), Europe (B.1), Alpha-UK (B.1.1.7), Beta S. Africa (B.1.351), Gamma Brazil (P.1), Delta India (B.1.617.2) and Omicron.

In specific embodiments, said immunogenic composition may further include one or more of the following excipients such as: a buffer, a salt, an osmolyte, an antioxidant and a surfactant or other agent to prevent protein loss on vial surfaces and/or protein aggregation.

The form of the pharmaceutical compositions, the route of administration, the dosage and the regimen naturally depend upon the condition to be treated, the severity of the illness, the age, weight, and sex of the patient, etc.

In preferred embodiments, the composition is formulated for intramuscular administration.

In some embodiments, the immunogenic composition is formulated in an effective amount of the N fusion protein (typically OVX033) to produce an antigen N-specific immune response in a subject with one or two doses, preferably administered via intramuscular route to a human subject.

For intramuscular administration, for example, the composition is an aqueous solution which should be suitably buffered if necessary and the liquid diluent first rendered at least isotonic with sufficient salts or sugars. In this connection, sterile aqueous media which can be employed will be known to those of skill in the art in light of the present disclosure. Examples of formulation for injectable solutions are provided in Remington: The Science and Practice of Pharmacy, 23^{rd} Edition, 2020. Some variation in dosage may occur depending on the condition of the subject being treated. Further specific examples of formulations are provided in the Examples, in particular for use with OVX033.

In specific embodiments, said immunogenic composition further comprises additional coronavirus antigens, typically selected from the Spike S protein or the Membrane M protein.

In specific embodiments, said immunogenic composition further comprises the nucleocapsid N antigen as the sole coronavirus antigen. In particular, it does not comprise additional coronavirus antigens selected from the Spike S protein or the membrane M protein.

In specific embodiments, said immunogenic composition further comprises one or more adjuvant, for example selected from the group consisting of aluminium salts (e.g. aluminium oxide, aluminium hydroxide or aluminium phosphate), squalene or liposome based adjuvants, optionally in combination with Toll Like Receptor (TLR) agonists or saponin.

In other specific embodiments, said immunogenic composition does not comprise any adjuvant.

In specific embodiments, the immunogenic composition is formulated as a ready-to-use sterile injectable solution.

Sterile injectable solutions are prepared by incorporating the active compound, i.e. the N fusion protein, in the required amount in the appropriate solvent with various of the other ingredients, as required, followed by filtered sterilization.

Methods of Use of the N Fusion Proteins and their Immunogenic Compositions

The N fusion proteins (in particular OVX033, and more specifically the N fusion protein as obtained from SEQ ID NO:11 in a bacterial host cell) and their immunogenic compositions as described in the previous sections are useful as a vaccine in the prevention of coronavirus disease, in particular COVID-19, typically for protection from severe COVID-19, in a human subject in need thereof.

Accordingly, the present disclosure provides compositions (e.g., immunogenic compositions as described in the previous section), methods, kits and reagents for prevention of coronavirus disease in humans and other mammals. The N fusion proteins or immunogenic compositions disclosed herein can be used as prophylactic agents. They may be used in medicine to prevent and/or protect from coronavirus disease, in particular COVID-19, more specifically to protect from severe COVID-19.

In exemplary aspects, the N fusion proteins or immunogenic compositions of the present disclosure are used to provide prophylactic protection from coronavirus disease, and in particular SARS-Cov2 disease, for example COVID-19, typically for protection from severe COVID-19. Prophylactic protection from coronavirus can be achieved following administration of an immunogenic amount of a composition of the present disclosure, typically with one or more immunogenic doses of OVX033. The immunogenic composition can be administered once, twice, three times, four times or more.

In some embodiments, the N fusion proteins or immunogenic compositions of the present disclosure can be used in a method of preventing a coronavirus infection in a subject, the method comprising administering to said subject at least one immunogenic dose of a N fusion protein or composition as provided herein, typically a composition comprising OVX033 as the the antigenic determinant.

In some embodiments, the N fusion proteins or immunogenic compositions of the present disclosure can be used as a method of inhibiting a primary coronavirus infection in a subject, the method comprising administering to said subject at least one immunogenic dose of a N fusion protein or composition as provided herein, typically a composition comprising OVX033 as the antigenic determinant.

In some embodiments, the N fusion proteins or immunogenic compositions of the present disclosure can be used as a method of reducing an incidence of coronavirus disease, typically coronavirus severe disease in a subject, the method comprising administering to said subject at least an immunogenic dose of a N fusion protein or composition as provided herein, typically a composition comprising OVX033 as the antigenic determinant. In some embodiments, the N fusion proteins or immunogenic compositions of the present disclosure can be used as a method of protecting from coronavirus disease, typically coronavirus severe disease such as severe COVID-19, in a subject, the method comprising administering to said subject at least an immunogenic dose of a N fusion protein or composition as provided herein, typically a composition comprising OVX033 as the antigenic determinant.

As used herein, the term “severe COVID-19” refers to either
Moderate Illness: Individuals who show evidence of lower respiratory disease during clinical assessment or imaging and who have an oxygen saturation (SpO2)≥94% on room air at sea level.
Severe Illness: Individuals who have SpO2<94% on room air at sea level, a ratio of arterial partial pressure of oxygen to fraction of inspired oxygen (PaO2/FiO2)<300 mm Hg, a respiratory rate >30 breaths/min, or lung infiltrates >50%.
Critical Illness: Individuals who have respiratory failure, septic shock, and/or multiple organ dysfunction.

In some embodiments, the term “severe COVID-19” refers to COVID-19 with respiratory deficiency symptoms requiring hospitalization.

In some embodiments, the N fusion proteins or immunogenic compositions of the present disclosure can be used as a method of protecting from one or more of severe symptoms of COVID-19, in a subject, the method comprising administering to said subject at least an immunogenic dose of a N fusion protein or composition as provided herein, typically a composition comprising OVX033 as the antigenic determinant. In some embodiments, the N fusion proteins or immunogenic composition of the present disclosure can be used as a method of inhibiting spread of coronavirus from a first subject infected with a coronavirus to a second subject not infected with coronavirus, the method comprising administering to said first subject and said second subject at least one immunogenic dose of a N fusion protein or composition as provided herein, typically a composition comprising OVX033 as the antigenic determinant.

As used herein, the term “immunogenic dose” refers to an amount specific to induce an antigen N specific immune response in a subject when administering such amount in a subject in need thereof, one or several times.

Some embodiments of the present disclosure provide methods of inducing an antigen N specific immune response in a subject, comprising administering to the subject any of the immunogenic compositions as provided herein (preferably an immunogenic composition with OVX033), in an amount effective to produce an N-specific immune response. In some embodiments, an antigen N specific immune response comprises total T cell response (in particular CD4+ or CD8+N specific T cell response) and/or a B cell response (specific anti-N immunoglobulin response).

Some embodiments of the present disclosure provide methods of inducing an antigen N specific cross-reactive immune response in a subject, comprising administering to the subject any of the immunogenic compositions as provided herein (preferably an immunogenic composition with Wuhan A SARS-Cov2 strain, and more preferably OVX033), in an amount effective to produce a cross-reactive N-specific immune response, in particular directed against other coronavirus strains, such as Europe (B.1), Alpha-UK (B.1.1.7), Beta S. Africa (B.1.351), Gamma Brazil (P.1), Delta India (B.1.617.2), Omicron, SARS-Cov1, and MERS-CoV.

Some embodiments of the present disclosure provide methods of inducing an antigen N specific cross-reactive immune response in a subject, comprising administering to the subject any of the immunogenic compositions as provided herein (preferably an immunogenic composition with Wuhan A SARS-Cov2 strain, and more preferably OVX033), in an amount effective to produce a cross-reactive N-specific immune response, in particular directed against other SARS-Cov2 strains, such as Europe (B.1), Alpha-UK (B.1.1.7), Beta S. Africa (B.1.351), Gamma Brazil (P.1), Delta India (B.1.617.2), and Omicron.

In some embodiments, an antigen N specific immune response comprises total T cell response (in particular CD4+ or CD8+N specific T cell response) and/or a B cell response (specific anti-N immunoglobulin response).

In some embodiments, a method of producing an antigen N-specific immune response comprises administering to a subject a single dose of an immunogenic composition of the present disclosure (typically with OVX033, for example as obtained from SEQ ID NO: 11, and/or from bacterial expression system such as E. coli host cell).

In some embodiments, the immunogenic composition (typically with OVX033) is administered to a subject by intradermal injection, intramuscular injection, or by intranasal administration. In preferred embodiments, the immunogenic composition (typically with OVX033) is administered to a subject by intramuscular injection.

The data presented in the Examples demonstrate significant increased protection against coronavirus disease using immunogenic compositions, in particular with OVX033 in hamster, as well as cross-reactive immune response to other coronavirus infection, such as other SARS-Cov2 strains.

In some embodiments, an effective amount of the N fusion protein (typically OVX033) is one or more doses of 10 μg to 1000 μg, 50 μg to 1000 μg, or more.

In some embodiments, the immunogenic composition (typically comprising OVX033) for use as a vaccine protects the subject against severe COVID-19.

In some embodiments, the immunogenic composition (typically comprising OVX033) for use as a vaccine protects the subject against one or more severe symptoms of coronavirus diseases, such as lung damages, respiratory failure, septic shock, and/or multiple organ dysfunction.

In some embodiments, the immunogenic composition with N fusion protein of SARS-Cov2 nucleocapsid N antigen (typically comprising OVX033) for use as a vaccine, provides cross-reactive immune response (e.g. CD4+ and/or CD8+ T-cell response specific to nucleocapsid N, anti-nucleocapsid N IgG (antibody response)) to other coronaviruses selected from the group consisting of Europe (B.1), Alpha-UK (B.1.1.7), Beta S. Africa (B.1.351), Gamma Brazil (P.1), Delta India (B.1.617.2). Omicron, SARS-Cov1, and MERS-COV.

In some embodiments, the immunogenic composition with N fusion protein of SARS-Cov2 nucleocapsid N antigen (typically comprising OVX033) for use as a vaccine, provides cross-reactive immune response (e.g. CD4+ and/or CD8+ T-cell response specific to nucleocapsid N, anti-nucleocapsid N IgG (antibody response)) to other SARS-Cov2 strains selected from the group consisting of Europe (B.1), Alpha-UK (B.1.1.7), Beta S. Africa (B.1.351), Gamma Brazil (P.1), Delta India (B.1.617.2), and Omicron,

In some embodiments, the immunogenic composition (typically comprising OVX033) for use as a vaccine, immunizes the subject against coronavirus disease from one or more of the SARS-Cov2 strains selected from the group consisting Wuhan (A), Europe (B.1), Alpha-UK (B.1.1.7), Beta S. Africa (B.1.351), Gamma Brazil (P.1), Delta India (B.1.617.2) and Omicron strains.

In some embodiments, the immunogenic composition (typically comprising OVX033) for use as a vaccine, provides cross-protection against one or more of coronavirus symptoms from other coronavirus disease, including protection against one or more of severe coronavirus symptoms, such as lung damages, respiratory failure, septic shock, and/or multiple organ dysfunction, and more specifically cross-protection from coronavirus disease resulting from a coronavirus infection selected from the group consisting of Europe (B.1), Alpha-UK (B.1.1.7), Beta S. Africa (B.1.351), Gamma Brazil (P.1), Delta India (B.1.617.2), Omicron, SARS-Cov1, and MERS-COV, more particularly selected from the group consisting of Europe B.1, delta, omicron and/or beta.

In some embodiments, the subject has been exposed to coronavirus (for example, one or more of the SARS-Cov2 strains selected from the group consisting of Wuhan (A), Europe (B.1), Alpha-UK (B.1.1.7). Beta S. Africa (B.1.351), Gamma Brazil (P.1), Delta India (B.1.617.2) and Omicron strain), the subject is infected with coronavirus, or the subject is at risk of infection by coronavirus.

In other embodiments, the subject is at risk of developing severe Covid-19. Risk factors include without limitation risk factors include (listed alphabetically) age (risk increases with each decade after age 50), cancer, cardiovascular disease, chronic kidney disease, chronic lung disease, diabetes, immunocompromising conditions or receipt of immunosuppressive medications, obesity (body mass index ≥30), pregnancy, and sickle cell disease.

In some embodiments, the subject is either (i) an elderly subject (e.g. older than 65 years, 70 years, or 80 years) (ii) a pregnant subject (iii) an immunocompromised subject or (iv) a child (e.g. a person younger than 18 years, 16, 14, 12, 10, 8, 6, 4, 2 years or younger).

In other embodiments, the immunogenic composition is administered in combination concomitantly or sequentially, with a second coronavirus vaccine, preferably selected from coronavirus vaccine including protein Spike or an antigenic fragment thereof.

In specific embodiments, the immunogenic composition is administered in combination concomitantly or sequentially, with a mRNA coronavirus vaccine, typically mRNA coronavirus including mRNA encoding SARS-Cov2 Spike protein or an antigenic fragment thereof, for example selected from the group consisting of COMIRNATY vaccine (Pfizer-BioNTech; BNT162b2 mRNA) or SPIKEVAX COVID-19 Vaccine (Moderna, mRNA-1273 SARS-COV-2).

As used herein, the term “combination”, “combined administration” or “concomitant administration” refers to a combined administration of at least two active ingredients (e.g. immunogenic compositions, where a first immunogenic composition comprising an N fusion protein as disclosed herein is administered at the same time or separately within time intervals, with a second vaccine or immunogenic composition, in the same subject in need thereof, where these time intervals allow that the combined active ingredients show a cooperative or synergistic effect for the immune response or protection against coronavirus, typically Covid-19. It is not intended to imply that the immunogenic compositions must be administered at the same time and/or formulated for delivery together although these methods of delivery are within the scope described herein. The terms are also meant to encompass regimens in which the active (immunogenic) agents are not necessarily administered by the same route of administration.

In specific embodiments, the other coronavirus vaccine (such as mRNA vaccine including mRNA encoding SARS-Cov2 Spike protein or an antigenic fragment) is first administered prior to said immunogenic composition of the present disclosure, typically within a time interval comprised between 1 and 3 months prior to administration of said immunogenic composition. In specific embodiments, the practitioner will adjust the sequence of administration of the vaccines ideally to have concomitant immune response of the other vaccine (such as mRNA vaccine) and the N fusion protein vaccine to produce a synergistic effect for the immune or protective response.

The disclosure will be further illustrated by the following embodiments, examples and figures. However, these examples and figures should not be interpreted in any way as limiting the scope of the present disclosure.

### SPECIFIC EMBODIMENTS

E1. An immunogenic composition for use as a vaccine for the prevention of coronavirus disease, in particular COVID-19 typically for protection from severe COVID-19, in a human subject in need thereof, wherein said immunogenic composition includes: a fusion protein comprising
   (i) a coronavirus nucleocapsid N antigen, preferably a SARS-Cov2 nucleocapsid N antigen, fused to
   (ii) a carrier protein comprising a self-assembling polypeptide derived from C4bp oligomerization domain and a positively charged tail.
E2. The immunogenic composition for use according to E1, wherein said fusion protein is not glycosylated.
E3. The immunogenic composition for use according to E1 or E2, wherein said fusion protein is obtainable from bacterial expression system, for example E. coli expression system.
E4. The immunogenic composition for use of any one of E1-E3, wherein the carrier protein is fused C-terminally to the nucleocapsid N antigen, optionally via a glycine-serine linker.
E5. The immunogenic composition for use of any one of E1-E4, wherein said fusion protein forms a heptameric protein after self-assembling.
E6. The immunogenic composition for use of any one of E1-E5, wherein said SARS-Cov2 nucleocapsid N antigen comprises at least one nucleocapsid N antigen from a SARS-Cov2 strain, for example, it essentially consists of the nucleocapsid N protein of SARS-Cov2 Wuhan (A) strain [PODTC9 (NCAP_SARS2), severe acute respiratory syndrome coronavirus 2 (2019-nCoV)].
E7. The immunogenic composition for use of any one of E1-E6, wherein said nucleocapsid N antigen comprises
   (i) a polypeptide of SEQ ID NO:1, or
   (ii) an antigenic polypeptide variant having at least 90% identity to SEQ ID NO:1.
E8. The immunogenic composition for use of any one of E1-E7, wherein said self-assembling polypeptide derived from C4bp oligomerization domain comprises SEQ ID NO:2, or a functional variant thereof having at least 90% identity to SEQ ID NO:2.
E9. The immunogenic composition for use of any one of E1-E8, wherein said positively charged tail comprises the sequence ZXBBBBZ (SEQ ID NO:3), wherein (i) Z is absent or is any amino acid, (ii) X is any amino acid, and (iii) B is an arginine or a lysine, preferably said positively charged tail comprises the sequence of SEQ ID NO:4.
E10. The immunogenic composition for use of any one of E1-E9, wherein said carrier protein essentially consists of SEQ ID NO:5, or said carrier protein is a functional variant of SEQ ID NO: 5 having at least 90% identity to SEQ ID NO:5.
E11. The immunogenic composition for use of any one of E1-E10, wherein said fusion protein comprises or essentially consists of SEQ ID NO:6, or is a functional variant of SEQ ID NO:6 having at least 90% identity to SEQ ID NO:6.
E12. The immunogenic composition for use of E11, wherein said fusion protein is encoded by SEQ ID NO:11.
E13. The immunogenic composition for use of any one of E1-E12, wherein said immunogenic composition is administered via intramuscular route.
E14. The immunogenic composition for use of any one of E1-E14, wherein said immunogenic composition does not comprise SARS-Cov2 spike S antigen, preferably, the SARS-Cov2 nucleocapsid N antigen is the only SARS-Cov2 antigenic determinant in the composition.
E15. The immunogenic composition for use of any one of E1-E14, wherein said use provides total T-cell response specific to nucleocapsid N, CD4+ and/or CD8+ T-cell response specific to nucleocapsid N, anti-nucleocapsid N IgG (antibody response) and/or protection against one or more of coronavirus symptoms, including protection against one or more of severe coronavirus symptoms, such as lung damages, respiratory failure, septic shock, and/or multiple organ dysfunction, and more specifically protection from a coronavirus disease due to infection with a coronavirus strain selected from the group consisting of Wuhan (A), Europe (B.1), Alpha-UK (B.1.1.7), Beta S. Africa (B.1.351), Gamma Brazil (P.1), Delta India (B.1.617.2), Omicron, SARS-Cov1, and MERS-COV, more particularly selected from the group consisting of Europe B.1, delta, omicron and/or beta.
E16. The immunogenic composition for use of any one of Claims E1-E15, wherein said SARS-Cov2 nucleocapsid N antigen is selected from Wuhan (A) strain, and said use provides total T-cell response specific to nucleocapsid N. CD4+/CD8+ T-cell response specific to nucleocapsid N, anti-nucleocapsid N IgG (antibody response) and/or cross-protection from coronavirus disease resulting from infection with a coronavirus strain selected from the group consisting of Europe (B.1), Alpha-UK (B.1.1.7), Beta S. Africa (B.1.351), Gamma Brazil (P.1). Delta India (B.1.617.2), Omicron, SARS-Cov1, and MERS-COV, more particularly selected from the group consisting of Europe (B.1), delta, omicron and/or beta strain.
E17. A fusion protein comprising
   (i) a SARS-Cov2 nucleocapsid N antigen fused to
   (ii) a carrier protein comprising a self-assembling polypeptide derived from C4bp oligomerization domain and a positively charged tail.
E18. The fusion protein of E17, which is not glycosylated.
E19. The fusion protein of E17 or E18, which is obtainable from bacterial expression system, for example E. coli expression system.
E20. The fusion protein of any one of E17-E19, wherein the carrier protein is fused C-terminally to the nucleocapsid N antigen, optionally via a glycine-serine linker.
E21. The fusion protein of any one of E17-E20, wherein said fusion protein forms a heptameric protein after self-assembling.
E22. The fusion protein of any one of E17-E21, wherein said SARS-Cov2 nucleocapsid N antigen comprises at least one nucleocapsid N antigen from a SARS-Cov2 strain Wuhan (A), typically strain [PODTC9 (NCAP_SARS2), severe acute respiratory syndrome coronavirus 2 (2019-nCOV)].
E23. The fusion protein of any one of E17-E22, wherein said nucleocapsid N antigen comprises
   (i) a polypeptide of SEQ ID NO:1, or
   (ii) an antigenic polypeptide variant having at least 90% identity to SEQ ID NO:1.
E24. The fusion protein of any one E17-E23, wherein said self-assembling polypeptide derived from C4bp oligomerization domain comprises SEQ ID NO:2, or a functional variant thereof having at least 90% identity to SEQ ID NO:2.
E25. The fusion protein of any one of E17-E24, wherein said positively charged tail comprises the sequence ZXBBBBZ (SEQ ID NO:3), wherein (i) Z is absent or is any amino acid, (ii) X is any amino acid, and (iii) B is an arginine or a lysine, preferably said positively charged tail comprises the sequence of SEQ ID NO:4.
E26. The fusion protein of any one of E17-E25, wherein said carrier protein essentially consists of SEQ ID NO:5, or said carrier protein is a functional variant of SEQ ID NO:5 having at least 90% identity to SEQ ID NO:5.
E27. The fusion protein of any one of E17-E26, wherein said fusion protein comprises or essentially consists of SEQ ID NO:6, or is a functional variant of SEQ ID NO:6 having at least 90% identity to SEQ ID NO:6.
E28. The fusion protein of E27, wherein said fusion protein is encoded by SEQ ID NO:11.
E29. A nucleic acid encoding a fusion protein as defined in any of E17-E28.
E30. An expression vector for the production of a fusion protein as defined in any of E17-E28, comprising a nucleic acid of E29.
E31. A host cell for the production of a fusion protein as defined in any of E17-E28, comprising an expression vector of E30.
E32. The host cell of E31, which is a bacteria, preferably E. coli host cell.
E33. An immunogenic composition comprising a fusion protein of any of E17-E28, and one or more pharmaceutically acceptable excipients.
E34. The immunogenic composition of E33, which is formulated for intramuscular administration.
E35. A process for producing a fusion protein of any one of E17-E28, said process comprising
   (i) culturing a host cell of E31 or E32 under conditions for synthesis of said fusion protein by said host cell,
   (ii) recovering said fusion protein, and,
   (iii) optionally, purifying said fusion protein.
E36. A method for inducing an immune response against coronavirus, typically SARS-Cov2, or preventing from coronavirus disease, in particular COVID-19 or severe COVID-19, in a human subject in need thereof, said method comprising administering an immunogenic amount of a composition comprising: a fusion protein comprising
   (i) a nucleocapsid N antigen, preferably SARS-Cov2 N antigen, fused to
   (ii) a carrier protein comprising a self-assembling polypeptide derived from C4bp oligomerization domain and a positively charged tail.
E37. The method of E36, wherein said fusion protein is not glycosylated.
E38. The method of E36 or E37, wherein said fusion protein is obtainable from bacterial expression system, for example E. coli expression system.
E39. The method of any one of E36-E38, wherein the carrier protein is fused C-terminally to the nucleocapsid N antigen, optionally via a glycine-serine linker.
E40. The method of of any one of E36-E39, wherein said fusion protein forms a heptameric particle after self-assembling.
E41. The method of any one of E36-E40, wherein said SARS-Cov2 nucleocapsid N antigen comprises at least one nucleocapsid N antigen from a SARS-Cov2 strain, for example, it essentially consists of the nucleocapsid N protein of SARS-Cov2 Wuhan (A) strain [PODTC9 (NCAP_SARS2), severe acute respiratory syndrome coronavirus 2 (2019-nCOV)]
E42. The method of any one of E36-E41, wherein said nucleocapsid N antigen comprises
   (i) a polypeptide of SEQ ID NO:1, or
   (ii) an antigenic polypeptide variant having at least 90% identity to SEQ ID NO:1.
E43. The method of any one of E36-E42, wherein said self-assembling polypeptide derived from C4bp oligomerization domain comprises SEQ ID NO:2, or a functional variant thereof having at least 90% identity to SEQ ID NO:2.
E44. The method of any one of E36-E43, wherein said positively charged tail comprises the sequence ZXBBBBZ (SEQ ID NO:3), wherein (i) Z is absent or is any amino acid, (ii) X is any amino acid, and (iii) B is an arginine or a lysine, preferably said positively charged tail comprises the sequence of SEQ ID NO:4.
E45. The method of any one of E36-E44, wherein said carrier protein essentially consists of SEQ ID NO:5, or said carrier protein is a functional variant of SEQ ID NO:5 having at least 90% identity to SEQ ID NO:5.
E46. The method of any one of E36-E45, wherein said fusion protein comprises or essentially consists of SEQ ID NO:6, or is a functional variant of SEQ ID NO:6 having at least 90% identity to SEQ ID NO:6.
E47. The method of E46, wherein said fusion protein is encoded by SEQ ID NO:11.
E48. The method of any one of E36-E47, wherein said immunogenic composition is administered by intramuscular injection.
E49. The method of any one of E36-E48, wherein said immunogenic composition does not comprise SARS-Cov2 spike antigen, preferably, the SARS-Cov2 nucleocapsid N antigen is the only SARS-Cov2 antigenic determinant in the composition.
E50. The method of any one of E36-E49, wherein said method provides total T-cell response specific to nucleocapsid N, CD4 T-cell or CD8+ T cell response specific to nucleocapsid N, anti-nucleocapsid N IgG (antibody response) and/or protection against one or more of coronavirus symptoms, including protection against one or more of severe coronavirus symptoms, such as lung damages, respiratory failure, septic shock, and/or multiple organ dysfunction, and more specifically protection or cross-protection from coronavirus disease due to an infection with a coronavirus strain selected from the group consisting of Wuhan (A). Europe (B.1), Alpha-UK (B.1.1.7), Beta S. Africa (B.1.351), Gamma Brazil (P.1), Delta India (B.1.617.2), Omicron, SARS-Cov1, and MERS-COV, more particularly selected from the group consisting of Europe B.1, delta, omicron and/or beta.
E51. The method of any one of E36-E49, wherein said SARS-Cov2 nucleocapsid N antigen is from the Wuhan (A) strain, and said method provides cross-reactive immune response to a coronavirus infection selected from the group consisting of Europe (B.1), Alpha-UK (B.1.1.7); Beta S. Africa (B.1.351), Gamma Brazil (P.1), Delta India (B.1.617.2), Omicron, SARS-Cov1, and MERS-COV. more particularly selected from the group consisting of Europe B.1, delta, omicron and/or beta.
E52. The method of any one of E36-E49, wherein said SARS-Cov2 nucleocapsid N antigen is from Wuhan A strain, and said method provides cross-protection against one or more of coronavirus symptoms from other coronavirus disease, including protection against one or more of severe coronavirus symptoms, such as lung damages, respiratory failure, septic shock, and/or multiple organ dysfunction, and more specifically cross-protection from coronavirus disease resulting from a coronavirus infection selected from the group consisting of Europe (B.1), Alpha-UK (B.1.1.7), Beta S. Africa (B.1.351), Gamma Brazil (P.1), Delta India (B.1.617.2), Omicron, SARS-Cov1, and MERS-COV, more particularly selected from the group consisting of Europe B.1, delta, omicron and/or beta.

### EXAMPLES

### Example 1: OVX030 Exhibits Truncated Form not Suitable for Vaccine Development

In order to produce the vaccine of the present disclosure, DNA constructs were obtained by DNA synthesis of E. coli codon optimized gene. First, two constructs were synthetized, one construct (SEQ ID NO:12) encoding OVX030 and another construct (SEQ ID NO:13) encoding OVX031 which consisted of the N of SARS-COV2 [PODTC9 (NCAP_SARS2), severe acute respiratory syndrome coronavirus 2 (2019-nCOV) (SARS-COV-2)] and the N of SARS-COV [P59595 (NCAP_SARS), severe acute respiratory syndrome coronavirus (SARS-COV)] fused to OVX313, respectively. Both constructs were cloned in an E. coli expression vector with IPTG-inducible T5 Promoter.

Protein expression of OVX030 and OVX031 encoding constructs using E. coli BL21 (New England BioLabs, #C2530H) allowed to reach high expression level even though the production was performed at 25° C. and in the presence of 30 μg/ml of kanamycin. Because its expression level was slightly higher than OVX031 and it consists of the N from the SARS-CoV2, the construct encoding OVX030 was firstly selected.

Small-scale batches of OVX030 were produced and initially purified by means of an ion exchange chromatography purification step followed by a size exclusion chromatography. These batches were subjected to a small set of analysis (concentration (A280), protein purity on SDS PAGE, Identity by Western Blotting, Intact mass of the monomer by LC-MS). The LC-MS analysis of a batch displayed an apparent mass of 51959.8 Da (A0.3 Da from the theorical MW) which complies with expectations.

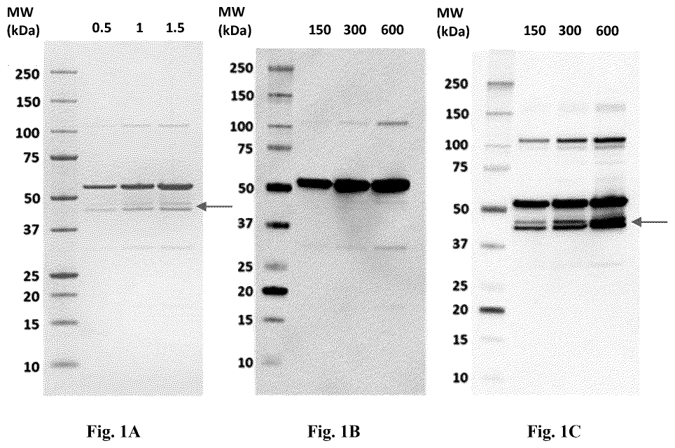

In contrast, the SDS PAGE and the Western Blot analysis (using an anti-nucleocapsid antibody) highlighted a truncated form of OVX030 that could not be removed during the purification steps (red narrow, FIG. 1). This finding was obviously not conformed to the features expected to the candidate vaccine.

### Example 2: Selection of a Codon-Optimized Sequence Suitable for Vaccine Development Encoding OVX033

Thereafter, three other constructs were synthesized. A first construct (SEQ ID NO:14) was synthesized encoding the N of the SARS-CoV2. OVX032 was produced to be used as a control protein without the OVX313 C-terminal domain. The two last constructs (encoding respectively OVX033 and OVX034) are two codon optimized variant sequences of the construct encoding N from SARS-COV2 fused to OVX313. The first one (SEQ ID NO:11) encoding OVX033 was a fully novel E. coli optimized gene, obtained by DNA synthesis. The second gene construct (SEQ ID NO: 15), encoding OVX034 was homologous to the gene construct encoding OVX032 for the DNA sequence corresponding to the nucleocapsid.

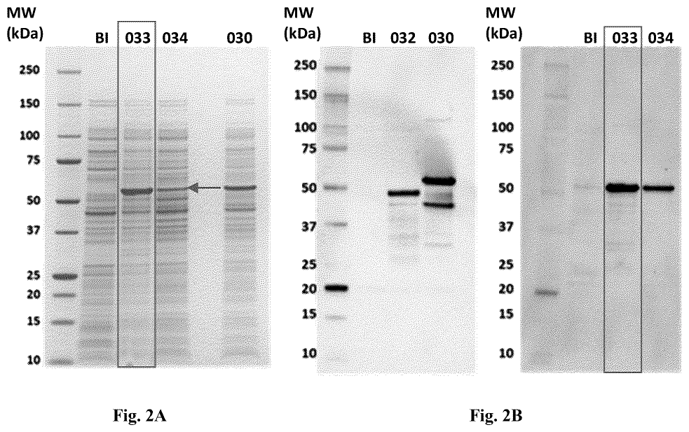

OVX033 construct, expressed in E. coli according to the parameters previously defined for OVX030 and OVX031, exhibit a high protein expression level with a substantially reduction of a truncated form expression (detected with OVX030 encoding construct). FIG. 2 illustrates the protein expression level of OVX033 and OVX034 compared to OVX030.

The OVX033 vaccine candidate is therefore a recombinant protein encoded by SEQ ID NO:11, as expressed in E. coli bacterial cell, and its subunit consists of the nucleocapsid (N) of SARS-CoV2 [PODTC9 (NCAP_SARS2), severe acute respiratory syndrome coronavirus 2 (2019-nCOV) (SARS-COV-2)] fused to OVX313 (Oligodom®). It is composed of 475 amino acids.

By means of the combination of disulfide bonds, hydrogen bonding, salt bridges and the large number of hydrophobic interactions, the OVX313 C-terminal domain enable the fused protein to heptamerize spontaneously: thus, OV033 contains 7 copies of each subunit and forms a heptameric recombinant protein with a molecular weight (MW) of 363.7 kDa (ie. 51.96 kDa×7) with an isoelectric point (pI) of 10.03.

### Example 3: Preclinical Data Using OVX033 in Hamster Protection Study

### 3.1 Study #1

Characteristics of OVX033 used in the first hamster protection study are presented below:

| | |
|---|---|
| Buffer | 50 mM sodium/potassium phosphate |
| | 375 mM sodium sulfate |
| | 200 mM trehalose |
| | 2 mM L-methionine |
| | 0.04% (v/v) polysorbate 80; [pH 6.9] |
| Storage | ~20° C. |
| Concentration | 2.51 mg/ml |
| (A280) | |
| Protein purity | >90% |
| (SDS-PAGE) | |
| Residual DNA | 18.7 ng/mg (ppm) |
| (qPCR | |
| Microbiology | 0 CFU/0.1 mg |
| control | |
| Endotoxin (LC-MS) | 11.9 ng/mg |

OVX033 was formulated at 1 mg/ml and study groups were:

| | ROUTE OF | |
|---|---|---|
| TEST ITEM | INJECTION | INJECTED VOLUME |
| OVX033 50 μg | IM | 50 μL |
| OVX033 50 μg | IN | 50 μL |
| NaCl | IM | 85 μL |

Ten hamsters/group were vaccinated intranasally (IN) or intramuscularly (IM) with OVX033 as described above, at DO and D28 (4 weeks apart). At D56, 28 days post last vaccinations, animal were intranasally infected using 10⁴ TCID₅₀ of Sars-COV-2 strain (Europe (B.1)). Five hamsters were sacrificed at D+4 post infection (D60), and the remaining hamsters (5 per group) were sacrificed at D+7 post infection (D63). Hamster weight was monitored daily from the day of infection. Viral loads were analyzed in lungs, throat swabs and in the nasal turbinates of every sacrificed hamsters at D60 and D63. Histopathological analyses were carried out on lungs collected from all sacrificed hamsters.

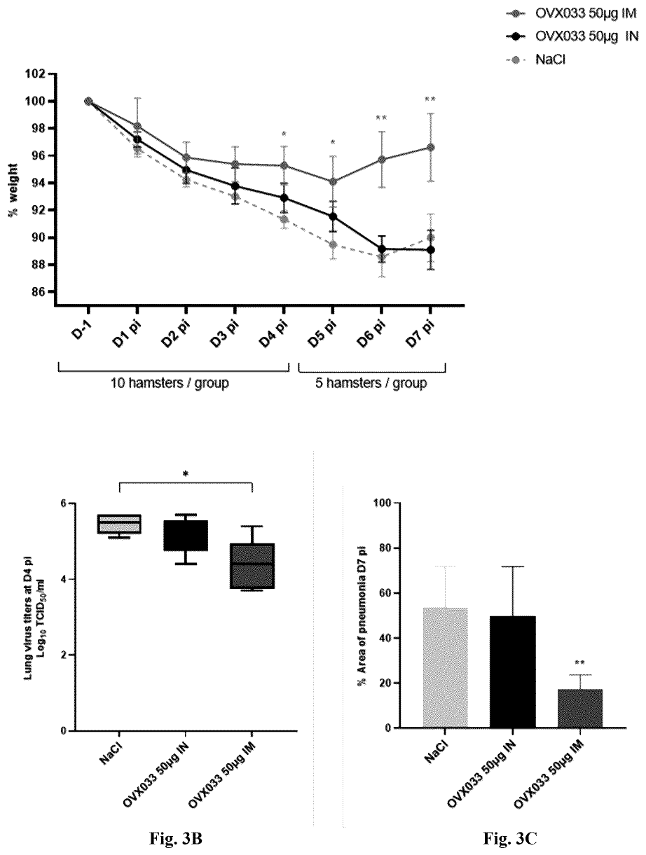

Key results obtained during the study are summarized hereafter:
About weight loss measured 7 days post Sars-COV-2 infection, OVX033 IM protected hamsters against weight loss following Sars-COV-2 infection, unlike OVX033 IN, with significant difference observed with the NaCl negative control group at D4, D5, D6 and D7 post infection (see FIG. 3A, Statistical analysis performed using 2-way ANOVA followed by Dunnett's multiple comparisons test);
a significant reduction of viral load measured in the lungs was observed only with OVX033 IM (see FIG. 3 B; statistical analysis: Mann Whitney tests between OVX033 treated group and NaCl group);
in the lungs, a significant reduction of lung damages was observed at D7 post infection with OVX033 IM (see FIG. 3 C, histopathology results, statistical analysis: t tests between OVX033 treated group and NaCl group).

### 3.2 Study #2

Characteristics of OVX033 used in the second hamster protection study are presented below:

| Buffer | 50 mM sodium/potassium phosphate | |
|---|---|---|
| | 375 mM sodium sulfate | |
| | 200 mM trehalose | |
| | 2 mM L-methionine | |
| | 0.04% (v/v) polysorbate 80; [pH | |
| | 6.91 | |
| Storage | −20° C. | |
| Concentration | 2.43 mg/mL | |
| (A280) | | |
| Protein purity | >90% | |
| (SDS-PAGE) | | |
| Residual DNA | 6 ng/mg (ppm) | |
| (qPCR) | | |
| Microbiology | 0 CFU/0.1 mg | |
| control | | |
| Endotoxin (LC-MS) | 10 ng/mg | |
| | ROUTE OF | |
| TEST ITEM | INJECTION | INJECTED VOLUME |
| OVX033 50 μg | IM | 100 μL |
| OVX033 100 μg | | 200 μL (2 sites x 100 μL) |
| NaCl | | 100 μL |

10 hamsters/group were vaccinated with the products described above, at DO and D28 (4 weeks apart). At D56, 28 days post last vaccinations, animal were intranasally infected using 10⁴ TCID₅₀ of Sars-COV-2 strain (Europe (B.1)). Five hamsters were sacrificed at D+4 post infection (D60), and the remaining hamsters (5 per group) were sacrificed at D+7 post infection (D63). Hamster weight was monitored daily from the day of infection. Viral loads was analyzed in lungs, throat swabs and in the nasal turbinates of every sacrificed hamsters at D60 and D63. Histopathological analyses were carried out on lungs collected from all sacrificed hamsters.

### Key Results Obtained During the Study:

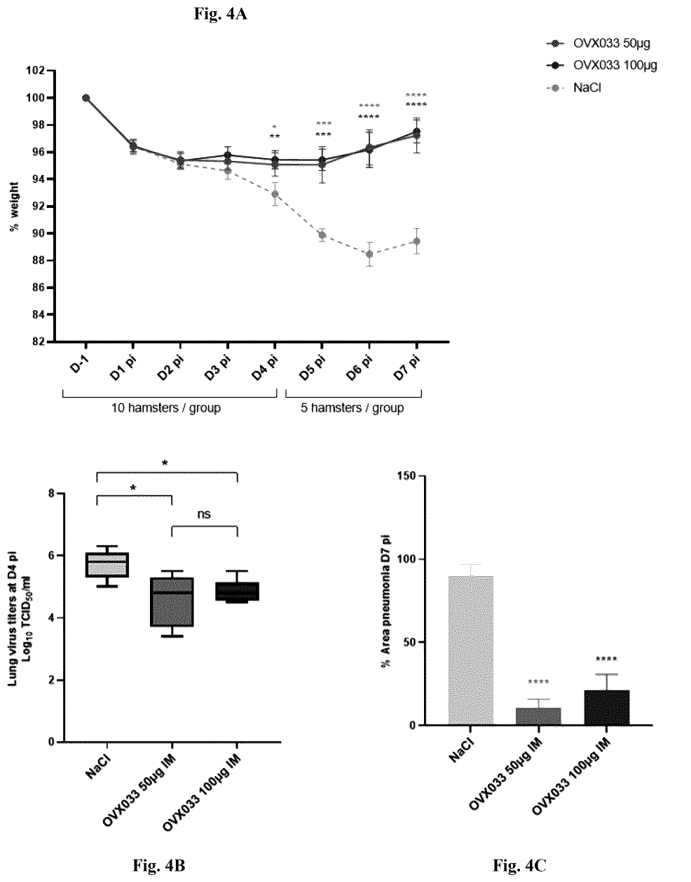

A significant reduction of viral load measured in the lungs was also observed with treated groups (OVX033 50 μg and 100 μg) (FIG. 4 B, statistical analysis: Mann-Whitney tests between OVX033 treated groups and NaCl group),
In the lungs, a significant reduction of lung damages was observed at D7 post infection with OVX033 50 μg and 100 μg (FIG. 4 C, histopathology results, statistical analysis: t tests between OVX033 treated groups and NaCl group).
About weight loss measured 7 days post SARS-COV-2 infection, OVX033 at 50 μg or 100 μg protected hamsters against weight loss following Sars-COV-2 infection, with significant difference observed with the NaCl negative control group at D4, D5, D6 and D7 post infection (FIG. 4A, Statistical analysis performed using 2-way ANOVA followed by Dunnett's multiple comparisons test).

### 3.3 Study #3

Characteristics of OVX033 used in the third hamster protection study are presented below:

| | |
|---|---|
| Buffer | 50 mM sodium/potassium phosphate |
| | 400 mM sodium sulfate |
| | 200 mM trehalose |
| | 2 mM L-methionine |
| | 0.04% (v/v) polysorbate 80 [pH 6.9] |
| Storage | −20° C. |
| Concentration (A280) | 2.18 mg/mL |
| Protein purity (SDS-PAGE) | >90% |
| Residual DNA (qPCR) | 29 ng/mg (ppm) |
| Microbiology control | 0 CFU/0.1 mg |
| Endotoxin (LAL) | 9 EU/mg |

OVX033 was formulated at 0.5 mg/ml:

| TEST ITEM | ROUTE OF INJECTION | INJECTED VOLUME |
|---|---|---|
| OVX033 50 μg | IM | 50 μL |

Twelve hamsters/group were vaccinated intramuscularly (IM) with OVX033 as described above, at D0 and D28 (4 weeks apart). At D56, 28 days post last vaccinations, animal were intranasally infected using 10⁴ TCID₅₀ of Sars-COV-2 strain (Delta, B.1.617.2). Six hamsters were sacrificed at D+4 post infection (D60), and the remaining hamsters (6 per group) were sacrificed at D+7 post infection (D63). Hamster weight was monitored daily from the day of infection. Viral loads were analyzed in lungs, throat swabs and in the nasal turbinates of every sacrificed hamsters at D60 and D63. Histopathological analyses were carried out on lungs collected from all sacrificed hamsters.

Key results obtained during the study are summarized hereafter:
a significant reduction of viral load measured by RT-qPCR in the lungs was observed after vaccination with OVX033 (see FIG. 5A: statistical analysis: Mann Whitney test between OVX033 treated group and NaCl group);
in the lungs, a significant reduction of lung damages was observed at D7 post infection with OVX033 (see FIG. 5 B, histopathology results, statistical analysis: Mann Whitney test between OVX033 treated group and NaCl group).

### 3.4 Study #4

Characteristics of OVX033 used in the fourth hamster protection study are presented below:

| | |
|---|---|
| Buffer | 50 mM sodium/potassium phosphate |
| | 400 mM sodium sulfate |
| | 200 mM trehalose |
| | 2 mM L-methionine |
| | 0.04% (v/v) polysorbate 80 [pH 6.9] |
| Storage | −20° C. |
| Concentration (A280) | 2.18 mg/mL |
| Protein purity (SDS-PAGE) | >90% |
| Residual DNA (qPCR) | 29 ng/mg (ppm) |
| Microbiology control | 0 CFU/0.1 mg |
| Endotoxin (LAL) | 9 EU/mg |

OVX033 was formulated at 0.5 mg/mL:

| TEST ITEM | ROUTE OF INJECTION | INJECTED VOLUME |
|---|---|---|
| OVX033 50 μg | IM | 50 μL |

Twelve hamsters/group were vaccinated intramuscularly (IM) with OVX033 as described above, at DO and D28 (4 weeks apart). At D56, 28 days post last vaccinations, animal were intranasally infected using 10⁴ TCID₅₀ of Sars-COV-2 strain (Omicron, B. 1.1.529). Six hamsters were sacrificed at D+4 post infection (D60), and the remaining hamsters (6 per group) were sacrificed at D+7 post infection (D63). Hamster weight was monitored daily from the day of infection. Viral loads were analyzed in lungs, throat swabs and in the nasal turbinates of every sacrificed hamsters at D60 and D63. Histopathological analyses were carried out on lungs collected from all sacrificed hamsters.

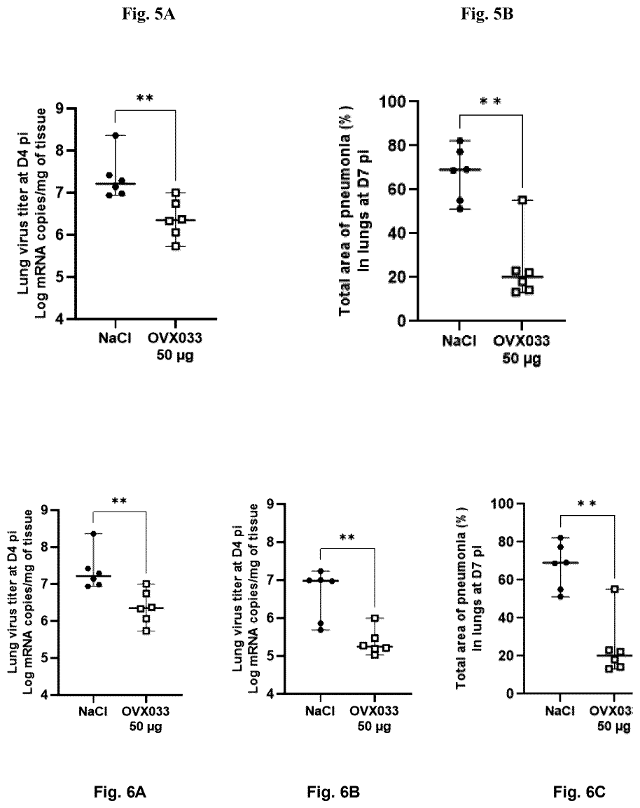

Key results obtained during the study are summarized hereafter:
a significant reduction of viral load measured by RT-qPCR in the lungs was observed after vaccination with OVX033 (see FIG. 6A; statistical analysis: Mann Whitney test between OVX033 treated group and NaCl group);
a significant reduction of viral load measured by RT-qPCR in nasal turbinates was observed 7 days after vaccination with OVX033 (see FIG. 6 B; statistical analysis: Mann Whitney test between OVX033 treated group and NaCl group);
in the lungs, a significant reduction of lung damages was observed at D7 post infection with OVX033 IM (see FIG. 6 C, histopathology results, statistical analysis: Mann Whitney test between OVX033 treated group and NaCl group).

### Example 4: Immunogenicity of OVX033 in Hamster

Characteristics of OVX033 used in this hamster immunogenicity study are presented below:

| | |
|---|---|
| Buffer | 50 mM sodium/potassium phosphate |
| | 400 mM sodium sulfate |
| | 200 mM trehalose |
| | 2 mM L-methionine |
| | 0.04% (v/v) polysorbate 80 [pH 6.9] |
| Storage | −20° C. |
| Concentration (A280) | 2.18 mg/mL |
| Protein purity (SDS-PAGE) | >90% |
| Residual DNA (qPCR) | 29 ng/mg (ppm) |
| Microbiology control | 0 CFU/0.1 mg |
| Endotoxin (LAL) | 9 EU/mg |

OVX033 was formulated at 0.5 mg/ml:

| TEST ITEM | ROUTE OF INJECTION | INJECTED VOLUME |
|---|---|---|
| OVX033 50 μg | IM | 50 μL |

Five hamsters/group were vaccinated intramuscularly (IM) with OVX033 as described above, at DO and D28 (4 weeks apart). Serum was collected from retro-orbital blood sampling at DO and D56 (sacrifice) for anti-N IgG analysis by ELISA. At D56, 28 days post last vaccinations, animal were euthanized, lungs and spleen were collected, cells isolated and analyzed by IFNγ ELISpot after 18 hours of stimulation with Nucleoprotein peptides pool (of Wuhan SARS-COV-2 strain, Swiss-Prot ID: PODTC9), 15 mers with 11 aa overlap.

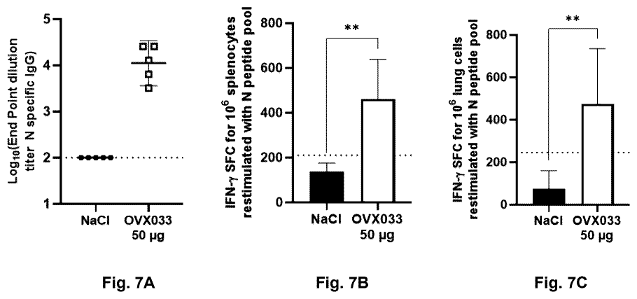

Key results obtained during the study are summarized hereafter:
High levels of anti-N specific serum IgG antibody were detected in sera after two vaccinations with OVX033 (see FIG. 7A, individual values and mean±95% CI).
a significant IFNγ T cells specific response was detected in spleen and lungs (see FIGS. 7B and 7 C; statistical analysis: Mann Whitney test between OVX033 treated group and NaCl group).

### Example 5: Comparative Study in Mice of OVX030 (Fusion N-OVX313) Vs OVX032 (N Antigen without Fusion) Showing IFNγ T Cell Specific Response in Lung Cells

### Materials &Methods:

OVX030 is a fusion of the N antigen of SARS-Cov2 with OVX313 and is described in Example 1 (encoded by SEQ ID NO:12). OVX032 is the N antigen of SARS-Cov2 as disclosed in Example 2 (encoded by SEQ ID NO:14).

5 groups of 5 C57BL6 mice were immunized at Day 0 and Day 28 with either of the five following regimen: (i) OVX032 (N); (ii) OVX030 (N-OVX313); (iii) OVX030 (N-OVX313)+Incomplete Freund's Adjuvant (IFA); (iv) OVX030 (N-OVX313)+MF59 Adjuvant (AddaVax) or (v) Buffer. The mice were sacrificed 8 days post-vaccination for evaluation of their immune response.

### Results:

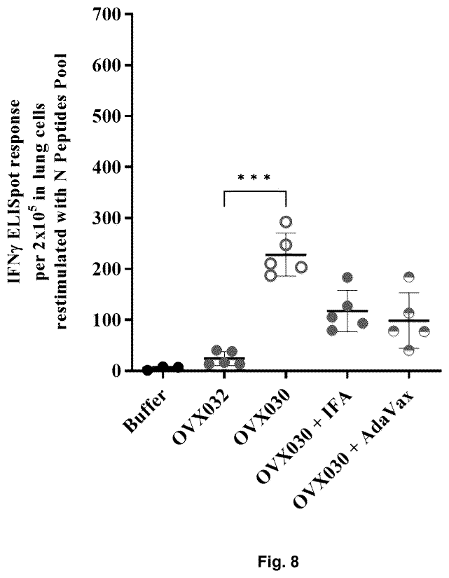

In lung cells, as shown in FIG. 8, OVX030 induces cellular responses. OVX032, the antigen N alone gave IFNγ cellular response significantly lower than OVX030 (N-OVX313), while the adjuvants tend to decrease the responses, but no statistical difference was found.

### Example 6: Useful Sequences for Practicing the Invention

TABLE 1
Brief description of the sequence

| SEQ ID | | |
|---|---|---|
| NO: | Type* | Brief Description |
| 1 | aa | Amino acid sequence of the |
| | | nucleocapsid N antigen (sequence as |
| | | used in OVX033 without methionine) |
| 2 | aa | Amino acid sequence of the hybrid |
| | | C4bp oligomerization domain |
| | | without the C-terminal positively |
| | | charged tail (including Glutamate as |
| | | last residue) |
| 3 | aa | Amino acid sequence of the |
| | | C-terminal positively charged tail |
| | | (ZXBBBBZ) |
| 4 | aa | Amino acid sequence of the |
| | | C-terminal positively charged tail |
| | | (GRRRRRS) |
| 5 | aa | OVX313 full amino acid sequence |
| | | without GS linker |
| 6 | aa | OVX033 full amino acid sequence |
| | | without methionine |
| 7 | nt | Nucleotide sequence encoding |
| | | the nucleocapsid antigen (sequence |
| | | as used in OVX033 without methionine) |
| 8 | nt | Nucleotide sequence encoding the |
| | | hybrid C4bp oligomerization |
| | | domain without the C-terminal |
| | | positively charged tail (including |
| | | Glutamate as last residue) |
| 9 | nt | Nucleotide sequence encoding the |
| | | C-terminal positively charged tail |
| | | (GRRRRRS) |
| 10 | nt | OVX313 full nucleotide coding |
| | | sequence without GS linker |
| 11 | nt | OVX033 full nucleotide coding |
| | | sequence without methionine |
| 12 | nt | OVX030 full nucleotide coding |
| | | sequence without methionine |
| 13 | nt | OVX031 full nucleotide coding |
| | | sequence without methionine |
| 14 | nt | OVX032 full nucleotide coding |
| | | sequence without methionine |
| 15 | nt | OVX034 full nucleotide coding |
| | | sequence without methionine |

*“aa” refers to amino acid and “nt” referes to nucleotide

TABLE 2
Sequence Listing

| | |
|---|---|
| 1 | SDNGPQNQRNAPRITFGGPSDSTGSNQNGERSGARSKQRRPQGLPNNTASWFTALT |
| | QHGKEDLKFPRGQGVPINTNSSPDDQIGYYRRATRRIRGGDGKMKDLSPRWYFYYLG |
| | TGPEAGLPYGANKDGIIWVATEGALNTPKDHIGTRNPANNAAIVLQLPQGTTLPKGFYA |
| | EGSRGGSQASSRSSSRSRNSSRNSTPGSSRGTSPARMAGNGGDAALALLLLDRLNQ |
| | LESKMSGKGQQQQGQTVTKKSAAEASKKPRQKRTATKAYNVTQAFGRRGPEQTQG |
| | NFGDQELIRQGTDYKHWPQIAQFAPSASAFFGMSRIGMEVTPSGTWLTYTGAIKLDDK |
| | DPNFKDQVILLNKHIDAYKTFPPTEPKKDKKKKADETQALPQRQKKQQTVTLLPAADLD |
| | DFSKQLQQSMSSADSTQA |
| 2 | KKQGDADVCGEVAYIQSVVSDCHVPTAELRTLLEIRKLFLEIQKLKVE |
| 3 | ZXBBBBZ |
| 4 | GRRRRRS |
| 5 | KKQGDADVCGEVAYIQSVVSDCHVPTAELRTLLEIRKLFLEIQKLKVEGRRRRRS |
| 6 | SDNGPQNQRNAPRITFGGPSDSTGSNQNGERSGARSKQRRPQGLPNNTASWFTALT |
| | QHGKEDLKFPRGQGVPINTNSSPDDQIGYYRRATRRIRGGDGKMKDLSPRWYFYYLG |
| | TGPEAGLPYGANKDGIIWVATEGALNTPKDHIGTRNPANNAAIVLQLPQGTTLPKGFYA |
| | EGSRGGSQASSRSSSRSRNSSRNSTPGSSRGTSPARMAGNGGDAALALLLLDRLNQ |
| | LESKMSGKGQQQQGQTVTKKSAAEASKKPRQKRTATKAYNVTQAFGRRGPEQTQG |
| | NFGDQELIRQGTDYKHWPQIAQFAPSASAFFGMSRIGMEVTPSGTWLTYTGAIKLDDK |
| | DPNFKDQVILLNKHIDAYKTFPPTEPKKDKKKKADETQALPQRQKKQQTVTLLPAADLD |
| | DFSKQLQQSMSSADSTQAGSKKQGDADVCGEVAYIQSVVSDCHVPTAELRTLLEIRKL |
| | FLEIQKLKVEGRRRRRS |
| 7 | AGCGACAATGGCCCGCAAAATCAACGCAACGCACCACGTATCACTTTCGGTGGCC |
| | CTAGCGATAGCACGGGTTCTAATCAGAATGGTGAACGTAGCGGTGCACGCAGCAA |
| | GCAACGTCGCCCGCAGGGCCTCCCGAACAATACGGCGTCTTGGTTCACCGCGCT |
| | GACGCAGCACGGTAAGGAAGATCTGAAATTTCCGCGTGGTCAGGGTGTTCCGATC |
| | AATACGAATAGCTCCCCGGACGATCAGATTGGCTATTACCGTCGTGCGACCCGTC |
| | GTATTCGTGGTGGCGACGGTAAGATGAAAGATCTGTCCCCACGCTGGTATTTCTAC |
| | TACCTGGGTACGGGCCCTGAGGCCGGTCTGCCGTATGGCGCGAACAAAGACGGC |
| | ATTATTTGGGTGGCGACCGAGGGTGCTCTGAACACGCCGAAAGACCACATCGGCA |
| | CCCGTAATCCGGCGAATAACGCAGCGATCGTTTTACAACTGCCGCAAGGTACGAC |
| | TCTGCCGAAGGGTTTTTACGCTGAGGGCAGCCGCGGTGGCAGCCAAGCGAGCAG |
| | CCGCAGCTCAAGCCGTAGCCGTAATAGCTCCAGAAACTCGACGCCTGGCAGCTCT |
| | CGTGGCACCAGCCCGGCACGTATGGCAGGCAACGGTGGCGATGCTGCGCTGGC |
| | CCTGCTGCTGCTTGATCGTCTGAACCAGCTGGAGTCCAAGATGAGCGGTAAGGGT |
| | CAGCAGCAGCAAGGTCAGACCGTGACCAAGAAATCTGCCGCAGAGGCGAGCAAG |
| | AAACCGCGTCAGAAGCGTACCGCGACCAAGGCCTACAACGTCACCCAAGCGTTTG |
| | GTCGCCGTGGTCCGGAGCAGACGCAAGGTAACTTCGGCGATCAAGAACTGATCC |
| | GCCAAGGCACCGACTACAAGCACTGGCCGCAGATCGCGCAATTTGCACCAAGCG |
| | CGTCGGCCTTTTTCGGTATGTCCCGCATTGGTATGGAAGTGACGCCGAGCGGTAC |
| | CTGGCTGACCTATACCGGTGCCATCAAACTGGATGATAAAGACCCGAACTTTAAAG |
| | ATCAAGTCATTTTGTTGAACAAGCATATTGACGCATATAAGACTTTCCCGCCGACC |
| | GAGCCGAAAAAAGACAAAAAGAAAAAAGCCGATGAAACCCAAGCACTGCCGCAGC |
| | GCCAAAAGAAACAGCAAACGGTTACCTTGCTGCCGGCTGCGGACCTGGACGACTT |
| | CAGCAAACAGTTGCAGCAGAGCATGAGCAGCGCTGACAGCACCCAGGCGTAA |
| 8 | AAGAAACAGGGTGATGCTGACGTGTGCGGCGAAGTGGCATATATCCAGAGCGTCG |
| | TGAGCGATTGTCACGTTCCGACGGCAGAGTTGCGCACGCTGTTGGAAATCCGTAA |
| | GCTGTTCTTGGAGATTCAAAAGCTCAAAGTTGAG |
| 9 | GGTCGTCGTCGCAGACGTTCC |
| 10 | AAGAAACAGGGTGATGCTGACGTGTGCGGCGAAGTGGCATATATCCAGAGCGTCG |
| | TGAGCGATTGTCACGTTCCGACGGCAGAGTTGCGCACGCTGTTGGAAATCCGTAA |
| | GCTGTTCTTGGAGATTCAAAAGCTCAAAGTTGAGGGTCGTCGTCGCAGACGTTCC |
| 11 | AGCGACAATGGCCCACAGAATCAACGTAATGCACCGAGAATCACCTTTGGCGGCC |
| | CGAGCGATAGCACGGGTAGCAACCAGAACGGCGAGCGCAGCGGTGCGCGTTCTA |
| | AACAGCGCCGTCCGCAAGGTCTGCCGAATAACACGGCGTCGTGGTTTACGGCACT |
| | GACCCAGCATGGTAAGGAAGATTTGAAGTTTCCGCGTGGCCAGGGTGTCCCGATC |
| | AATACCAACAGCTCACCTGATGACCAGATCGGCTATTATCGTCGCGCCACGCGCC |
| | GTATCCGCGGTGGCGATGGCAAGATGAAAGACTTAAGCCCGCGTTGGTACTTCTA |
| | TTACCTGGGTACGGGTCCAGAGGCTGGTTTGCCGTATGGTGCTAACAAAGACGGC |
| | ATTATCTGGGTGGCGACCGAGGGTGCCCTGAACACCCCGAAAGATCACATTGGTA |
| | CTCGTAACCCTGCGAACAACGCCGCGATTGTGCTGCAACTGCCGCAGGGTACCAC |
| | CCTGCCGAAGGGTTTCTATGCAGAGGGCAGCCGTGGTGGCAGCCAGGCGAGCAG |
| | CCGTAGCAGCAGCCGTTCCCGCAATTCTAGCCGCAATAGCACGCCGGGTTCCAGC |
| | CGTGGTACGTCCCCGGCACGTATGGCAGGCAATGGTGGCGATGCGGCACTGGCG |
| | CTGCTGTTGCTGGACCGCCTGAACCAACTGGAGAGCAAGATGAGCGGCAAGGGT |
| | CAGCAGCAGCAAGGTCAGACTGTGACCAAAAAGTCCGCGGCAGAGGCCAGCAAG |
| | AAACCACGTCAGAAACGTACCGCGACCAAAGCGTACAATGTTACCCAGGCCTTCG |
| | GTCGCCGTGGCCCTGAGCAAACCCAAGGTAATTTCGGTGACCAAGAACTGATCCG |
| | CCAGGGCACGGACTACAAGCATTGGCCGCAGATTGCGCAGTTTGCACCGAGCGC |
| | TAGCGCGTTCTTCGGTATGTCCCGCATTGGTATGGAAGTGACCCCGTCTGGCACC |
| | TGGCTGACCTACACTGGTGCAATCAAATTGGACGATAAAGACCCGAACTTTAAAGA |
| | CCAAGTCATCCTCCTGAACAAGCACATTGATGCGTACAAGACCTTCCCGCCGACG |
| | GAACCGAAAAAAGACAAAAAGAAAAAAGCTGACGAAACCCAAGCATTGCCGCAAC |
| | GTCAAAAGAAACAGCAAACGGTTACCCTGCTTCCGGCAGCGGATCTGGACGATTT |
| | TAGCAAGCAACTGCAACAATCTATGTCGAGCGCGGACTCCACCCAGGCCGGTAGC |
| | AAGAAACAGGGCGATGCCGATGTCTGCGGCGAAGTTGCGTACATTCAGAGCGTCG |
| | TGAGCGACTGTCACGTTCCGACGGCTGAGCTGCGTACCCTGCTGGAAATTCGTAA |
| | GCTGTTCCTGGAAATCCAAAAGCTGAAAGTTGAGGGTCGCCGTCGTCGCCGTAGC |
| | TAA |
| 12 | AGCGATAATGGCCCACAAAATCAACGTAACGCACCAAGAATCACCTTTGGCGGCC |
| | CGAGCGACTCCACCGGTTCGAATCAGAATGGTGAGCGTAGCGGTGCACGCAGCA |
| | AGCAACGCCGTCCGCAGGGCTTACCGAACAACACGGCTTCCTGGTTCACCGCGCT |
| | GACTCAGCACGGTAAAGAGGACCTGAAGTTTCCTCGTGGTCAGGGTGTGCCGATC |
| | AACACCAACAGCTCCCCGGATGACCAGATCGGTTACTATCGCCGTGCAACCCGCC |
| | GTATTCGTGGTGGCGACGGTAAGATGAAAGATCTGAGCCCGCGTTGGTATTTCTAT |
| | TACCTGGGTACGGGTCCGGAAGCGGGCCTGCCGTACGGTGCGAACAAAGACGGC |
| | ATCATTTGGGTTGCGACCGAGGGTGCGCTGAATACGCCGAAAGATCACATTGGTA |
| | CCCGCAACCCGGCTAATAACGCCGCGATCGTCTTGCAGCTGCCGCAGGGTACCA |
| | CCCTGCCGAAGGGCTTCTATGCCGAGGGTAGCCGCGGTGGCAGCCAAGCGTCTA |
| | GCCGTAGCAGCAGCCGTAGCCGTAATAGCTCCCGTAATAGCACGCCGGGTAGCA |
| | GCCGTGGCACGAGCCCGGCTCGCATGGCAGGCAATGGCGGCGATGCGGCGCTT |
| | GCACTGCTGCTGCTGGACCGTCTGAACCAACTGGAGAGCAAGATGAGCGGCAAG |
| | GGCCAGCAACAACAGGGTCAGACCGTCACCAAGAAAAGCGCAGCCGAGGCGTCT |
| | AAAAAGCCGCGTCAGAAGCGCACGGCGACGAAAGCATACAATGTGACGCAAGCG |
| | TTCGGTCGTCGCGGTCCGGAGCAGACCCAGGGTAACTTCGGCGACCAAGAACTC |
| | ATCCGTCAAGGTACCGATTACAAGCATTGGCCACAGATTGCGCAGTTTGCTCCGA |
| | GCGCTTCCGCATTTTTCGGCATGTCTCGTATCGGTATGGAAGTGACCCCGTCCGG |
| | TACCTGGCTGACTTATACGGGTGCCATCAAACTGGATGATAAAGATCCTAACTTTA |
| | AAGACCAAGTGATTTTGTTGAACAAGCACATTGACGCGTACAAGACCTTCCCGCCG |
| | ACCGAGCCGAAAAAAGACAAGAAAAAAAAAGCCGATGAAACCCAAGCGCTGCCGC |
| | AGCGCCAAAAGAAACAACAAACTGTCACGCTGCTGCCTGCGGCGGACCTGGACG |
| | ATTTTTCGAAGCAACTGCAACAGAGCATGAGCAGCGCGGACTCAACCCAGGCCGG |
| | CAGCAAGAAACAGGGTGATGCAGATGTTTGTGGCGAAGTCGCCTACATTCAGTCT |
| | GTTGTGAGCGACTGCCATGTTCCGACCGCAGAGCTGCGCACGCTGTTGGAAATTC |
| | GTAAGCTGTTCTTGGAAATCCAGAAACTGAAAGTTGAGGGTCGTCGTCGTCGCCG |
| | CAGC |
| 13 | TCTGATAATGGCCCGCAATCCAACCAACGTTCTGCACCGCGCATCACTTTCGGTG |
| | GCCCGACCGACAGCACGGATAACAACCAGAACGGTGGCCGCAACGGTGCACGTC |
| | CGAAACAGCGTCGTCCGCAGGGTCTGCCGAACAATACCGCCTCTTGGTTCACCGC |
| | GCTGACGCAGCATGGCAAGGAAGAACTGCGTTTCCCGCGTGGTCAGGGTGTACC |
| | GATTAACACGAATAGCGGTCCTGATGATCAAATCGGCTACTATCGTCGTGCGACCC |
| | GTCGTGTGCGCGGTGGCGACGGCAAGATGAAAGAACTGTCCCCACGCTGGTACT |
| | TTTACTATCTGGGCACCGGTCCGGAAGCGTCCCTGCCGTACGGTGCTAATAAAGA |
| | GGGCATTGTTTGGGTTGCGACTGAGGGTGCGTTGAACACCCCGAAAGACCACATC |
| | GGTACCCGTAATCCGAACAATAACGCAGCGACGGTGCTGCAACTGCCGCAGGGC |
| | ACGACGCTGCCGAAGGGTTTCTACGCCGAGGGTAGCCGTGGTGGCTCGCAGGCG |
| | AGCAGCCGCTCTAGCAGCCGTAGCCGCGGTAATAGCCGCAATAGCACCCCTGGC |
| | AGCAGCCGTGGTAATTCCCCTGCGCGTATGGCGAGCGGTGGCGGCGAAACGGCG |
| | CTGGCGCTGCTCCTGCTGGATCGTTTGAACCAGTTGGAGAGCAAGGTTAGCGGTA |
| | AAGGCCAACAGCAGCAAGGTCAAACCGTCACCAAAAAGTCAGCAGCCGAGGCAA |
| | GCAAAAAGCCACGTCAGAAGCGCACGGCTACGAAGCAATACAATGTCACCCAAGC |
| | CTTTGGCCGTCGCGGTCCGGAGCAAACGCAAGGTAACTTTGGCGATCAAGACCTG |
| | ATTCGCCAGGGTACCGACTATAAGCACTGGCCGCAAATCGCTCAATTCGCCCCGA |
| | GCGCCTCCGCGTTCTTCGGTATGAGCCGCATTGGTATGGAAGTGACCCCGAGCG |
| | GTACCTGGCTGACCTATCATGGCGCGATCAAACTGGATGATAAAGACCCGCAGTT |
| | TAAAGACAACGTTATCCTGTTAAACAAGCACATCGATGCTTACAAGACCTTTCCACC |
| | GACCGAGCCGAAAAAAGACAAAAAGAAAAAGACCGACGAAGCGCAGCCGCTGCC |
| | GCAGCGCCAGAAGAAACAACCGACCGTTACTCTTCTGCCGGCCGCAGACATGGAT |
| | GACTTTAGCCGCCAACTGCAGAATTCGATGAGCGGTGCAAGCGCGGATAGCACCC |
| | AGGCTGGCAGCAAGAAACAGGGTGACGCAGATGTGTGCGGCGAAGTCGCGTATA |
| | TTCAAAGCGTGGTTAGCGACTGTCACGTGCCGACCGCAGAGTTGCGTACGCTGCT |
| | GGAAATTCGTAAGCTGTTCCTGGAAATTCAGAAACTGAAAGTCGAGGGTCGTCGTC |
| | GTAGACGTAGC |
| 14 | AGCGACAATGGCCCGCAAAATCAACGCAACGCACCACGTATCACTTTCGGTGGCC |
| | CTAGCGATAGCACGGGTTCTAATCAGAATGGTGAACGTAGCGGTGCACGCAGCAA |
| | GCAACGTCGCCCGCAGGGCCTCCCGAACAATACGGCGTCTTGGTTCACCGCGCT |
| | GACGCAGCACGGTAAGGAAGATCTGAAATTTCCGCGTGGTCAGGGTGTTCCGATC |
| | AATACGAATAGCTCCCCGGACGATCAGATTGGCTATTACCGTCGTGCGACCCGTC |
| | GTATTCGTGGTGGCGACGGTAAGATGAAAGATCTGTCCCCACGCTGGTATTTCTAC |
| | TACCTGGGTACGGGCCCTGAGGCCGGTCTGCCGTATGGCGCGAACAAAGACGGC |
| | ATTATTTGGGTGGCGACCGAGGGTGCTCTGAACACGCCGAAAGACCACATCGGCA |
| | CCCGTAATCCGGCGAATAACGCAGCGATCGTTTTACAACTGCCGCAAGGTACGAC |
| | TCTGCCGAAGGGTTTTTACGCTGAGGGCAGCCGCGGTGGCAGCCAAGCGAGCAG |
| | CCGCAGCTCAAGCCGTAGCCGTAATAGCTCCAGAAACTCGACGCCTGGCAGCTCT |
| | CGTGGCACCAGCCCGGCACGTATGGCAGGCAACGGTGGCGATGCTGCGCTGGC |
| | CCTGCTGCTGCTTGATCGTCTGAACCAGCTGGAGTCCAAGATGAGCGGTAAGGGT |
| | CAGCAGCAGCAAGGTCAGACCGTGACCAAGAAATCTGCCGCAGAGGCGAGCAAG |
| | AAACCGCGTCAGAAGCGTACCGCGACCAAGGCCTACAACGTCACCCAAGCGTTTG |
| | GTCGCCGTGGTCCGGAGCAGACGCAAGGTAACTTCGGCGATCAAGAACTGATCC |
| | GCCAAGGCACCGACTACAAGCACTGGCCGCAGATCGCGCAATTTGCACCAAGCG |
| | CGTCGGCCTTTTTCGGTATGTCCCGCATTGGTATGGAAGTGACGCCGAGCGGTAC |
| | CTGGCTGACCTATACCGGTGCCATCAAACTGGATGATAAAGACCCGAACTTTAAAG |
| | ATCAAGTCATTTTGTTGAACAAGCATATTGACGCATATAAGACTTTCCCGCCGACC |
| | GAGCCGAAAAAAGACAAAAAGAAAAAAGCCGATGAAACCCAAGCACTGCCGCAGC |
| | GCCAAAAGAAACAGCAAACGGTTACCTTGCTGCCGGCTGCGGACCTGGACGACTT |
| | CAGCAAACAGTTGCAGCAGAGCATGAGCAGCGCTGACAGCACCCAGGCGTAA |
| 15 | AGCGACAATGGCCCGCAAAATCAACGCAACGCACCACGTATCACTTTCGGTGGCC |
| | CTAGCGATAGCACGGGTTCTAATCAGAATGGTGAACGTAGCGGTGCACGCAGCAA |
| | GCAACGTCGCCCGCAGGGCCTCCCGAACAATACGGCGTCTTGGTTCACCGCGCT |
| | GACGCAGCACGGTAAGGAAGATCTGAAATTTCCGCGTGGTCAGGGTGTTCCGATC |
| | AATACGAATAGCTCCCCGGACGATCAGATTGGCTATTACCGTCGTGCGACCCGTC |
| | GTATTCGTGGTGGCGACGGTAAGATGAAAGATCTGTCCCCACGCTGGTATTTCTAC |
| | TACCTGGGTACGGGCCCTGAGGCCGGTCTGCCGTATGGCGCGAACAAAGACGGC |
| | ATTATTTGGGTGGCGACCGAGGGTGCTCTGAACACGCCGAAAGACCACATCGGCA |
| | CCCGTAATCCGGCGAATAACGCAGCGATCGTTTTACAACTGCCGCAAGGTACGAC |
| | TCTGCCGAAGGGTTTTTACGCTGAGGGCAGCCGCGGTGGCAGCCAAGCGAGCAG |
| | CCGCAGCTCAAGCCGTAGCCGTAATAGCTCCAGAAACTCGACGCCTGGCAGCTCT |
| | CGTGGCACCAGCCCGGCACGTATGGCAGGCAACGGTGGCGATGCTGCGCTGGC |
| | CCTGCTGCTGCTTGATCGTCTGAACCAGCTGGAGTCCAAGATGAGCGGTAAGGGT |
| | CAGCAGCAGCAAGGTCAGACCGTGACCAAGAAATCTGCCGCAGAGGCGAGCAAG |
| | AAACCGCGTCAGAAGCGTACCGCGACCAAGGCCTACAACGTCACCCAAGCGTTTG |
| | GTCGCCGTGGTCCGGAGCAGACGCAAGGTAACTTCGGCGATCAAGAACTGATCC |
| | GCCAAGGCACCGACTACAAGCACTGGCCGCAGATCGCGCAATTTGCACCAAGCG |
| | CGTCGGCCTTTTTCGGTATGTCCCGCATTGGTATGGAAGTGACGCCGAGCGGTAC |
| | CTGGCTGACCTATACCGGTGCCATCAAACTGGATGATAAAGACCCGAACTTTAAAG |
| | ATCAAGTCATTTTGTTGAACAAGCATATTGACGCATATAAGACTTTCCCGCCGACC |
| | GAGCCGAAAAAAGACAAAAAGAAAAAAGCCGATGAAACCCAAGCACTGCCGCAGC |
| | GCCAAAAGAAACAGCAAACGGTTACCTTGCTGCCGGCTGCGGACCTGGACGACTT |
| | CAGCAAACAGTTGCAGCAGAGCATGAGCAGCGCTGACAGCACCCAGGGGGGCAG |
| | CAAGAAACAGGGTGACGCAGATGTGTGCGGCGAAGTCGCGTATATTCAAAGCGTG |
| | GTTAGCGACTGTCACGTGCCGACCGCAGAGTTGCGTACGCTGCTGGAAATTCGTA |
| | AGCTGTTCCTGGAAATTCAGAAACTGAAAGTCGAGGGTCGTCGTCGTAGACGTAG |
| | CTAA |

1. An immunogenic composition comprising a fusion protein that comprises
(i) a SARS-Cov2 nucleocapsid N antigen fused to
(ii) a carrier protein comprising a self-assembling polypeptide derived from C4bp oligomerization domain and a positively charged tail,
wherein said SARS-Cov2 nucleocapsid N antigen is the nucleocapsid N antigen of Wuhan (A) strain.

2. The immunogenic composition of claim 1, wherein said fusion protein is not glycosylated.

3. (canceled)

4. The immunogenic composition of claim 1, wherein the carrier protein is fused C-terminally to the nucleocapsid N antigen, optionally via a glycine-serine linker.

5. (canceled)

6. (canceled)

7. The immunogenic composition of claim 1, wherein said nucleocapsid N antigen comprises
(i) a polypeptide of SEQ ID NO:1, or
(ii) an antigenic polypeptide variant having at least 90% identity to SEQ ID NO:1.

8. The immunogenic composition of claim 1, wherein said self-assembling polypeptide derived from C4bp oligomerization domain comprises SEQ ID NO:2, or a functional variant thereof having at least 90% identity to SEQ ID NO:2.

9. The immunogenic composition of claim 1, wherein said positively charged tail comprises the sequence ZXBBBBZ (SEQ ID NO: 3), wherein (i) Z is absent or is any amino acid, (ii) X is any amino acid, and (iii) B is an arginine or a lysine, preferably said positively charged tail comprises the sequence of SEQ ID NO:4.

10. The immunogenic composition of claim 1, wherein said carrier protein essentially consists of SEQ ID NO:5, or said carrier protein is a functional variant of SEQ ID NO:5 having at least 90% identity to SEQ ID NO: 5.

11. The immunogenic composition of claim 1, wherein said fusion protein comprises or essentially consists of SEQ ID NO:6, or is a functional variant of SEQ ID NO:6 having at least 90% identity to SEQ ID NO:6.

12. The immunogenic composition for use of claim 11, wherein said fusion protein is encoded by SEQ ID NO:11.

13. (canceled)

14. (canceled)

15. (canceled)

16. (canceled)

17. A method for the prevention of coronavirus disease in a human subject in need thereof, said method comprising administering an efficient amount of an immunogenic composition of claim 1 to said subject.

18. The method of claim 17, which provides cross-protection to coronavirus disease resulting from a coronavirus infection by a coronavirus strain other than Wuhan (A) strain.

19. The method of claim 17, wherein said other coronavirus strain is selected from SARS Cov, SARS-Cov2 or MERS-Cov strain.

20. The method of claim 17, wherein aid immunogenic composition is administered via intramuscular route.

21. The method of claim 17, which provides total T-cell response specific to nucleocapsid N, CD4+/CD8+ T-cell response specific to nucleocapsid N, and/or anti-nucleocapsid N IgG (antibody response).

22. The method of claim 17, which provides cross-protection against one or more of severe coronavirus symptoms, such as lung damages, respiratory failure, septic shock, and/or multiple organ dysfunction.

23. The method of claim 17, which provides cross-protection to COVID19.

24. The method of claim 17, which provides cross-protection to severe COVID19.

25. The method of claim 17, which provides cross-protection to coronavirus disease resulting from a SARS-Cov2 infection selected from the group consisting of Europe (B.1), delta, omicron, and/or beta strain.

26. The method of claim 17, wherein said immunogenic composition is administered in combination, concomitantly or sequentially, with a second coronavirus vaccine composition including protein Spike or an antigenic fragment thereof, or an mRNA vaccine comprising an mRNA encoding SRS-Cov2 protein Spike, or an antigen fragment thereof.

27. The method of claim 26, wherein said second coronavirus vaccine composition is selected from COMIRNATY vaccine (Pfizer-BioNTech; BNT162b2 mRNA) and SPIKEVAX COVID-19 Vaccine (Moderna, mRNA-1273 SARS-COV-2).

28. A method of inducing an antigen N specific cross-reactive immune response in a subject in need thereof, said method comprising administering to a subject an amount of an immunogenic composition of claim 1.
